(19) United States
(12) Patent Application Publication
(10) Pub. No.: US 2007/0233246 A1
(43) Pub. Date: Oct. 4, 2007

(54) Spinal implants with improved mechanical response

(75) Inventors: Hai H. Trieu, Cordova, TN (US); Fred J. Molz IV, Birmingham, AL (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/396,253

(22) Filed: Mar. 31, 2006

(51) Int. Cl. A61F 2/44

(52) U.S. Cl. 623/17.11; 623/926

(57) ABSTRACT

A method of treating a patient includes determining a patient characteristic associated with the patient, determining a property value based at least in part on the patient characteristic, and determining a crosslinking parameter based at least in part on the property value.

### FIELD OF THE DISCLOSURE

The present disclosure relates generally to orthopedic and spinal devices. More specifically, the present disclosure relates to spinal implants.

### BACKGROUND

In human anatomy, the spine is a generally flexible column that can take tensile and compressive loads. The spine also allows bending motion and provides a place of attachment for keels, muscles and ligaments. Generally, the spine is divided into four sections: the cervical spine, the thoracic or dorsal spine, the lumbar spine, and the pelvic spine. The pelvic spine generally includes the sacrum and the coccyx. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other, particularly during bending, or flexure, of the spine. Thus, the intervertebral discs are under constant muscular and gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints or intervertebral disc may cause spinal stenosis, degenerative spondylolisthesis, and degenerative scoliosis.

One surgical procedure for treating these conditions is spinal arthrodesis, i.e., vertebral fusion, which can be performed anteriorally, posteriorally, or laterally. The posterior procedures include in-situ fusion, posterior lateral instrumented fusion, transforaminal lumbar interbody fusion (“TLIF”) or posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertebrae;

FIG. 3 is a top plan view of a vertebra;

FIG. 4 is a cross section view of an intervertebral disc;

FIGS. 5 and 6 are flow charts including illustrations of exemplary methods for treating a patient.

FIGS. 7A, 7B, 7C, and 7D are cross-sectional views of an exemplary component for use in an implantable device.

FIGS. 8 and 9 include illustrations of exemplary systems for forming a medical device.

FIG. 10 is an anterior view of a first embodiment of an intervertebral prosthetic disc;

FIG. 11 is an exploded anterior view of the first embodiment of the intervertebral prosthetic disc;

FIG. 12 is a further view of the first embodiment of the intervertebral prosthetic disc;

FIG. 13 is a lateral view of the first embodiment of the intervertebral prosthetic disc;

FIG. 14 is an exploded lateral view of the first embodiment of the intervertebral prosthetic disc;

FIG. 15 is a plan view of a superior half of the first embodiment of the intervertebral prosthetic disc;

FIG. 16 is a plan view of an inferior half of the first embodiment of the intervertebral prosthetic disc;

FIG. 17 is an exploded lateral view of the first embodiment of the intervertebral prosthetic disc installed within an intervertebral space between a pair of adjacent vertebrae;

FIG. 18 is an anterior view of the first embodiment of the intervertebral prosthetic disc installed within an intervertebral space between a pair of adjacent vertebrae;

FIG. 19 is a posterior view of a second embodiment of an intervertebral prosthetic disc;

FIG. 20 is an exploded posterior view of the second embodiment of the intervertebral prosthetic disc;

FIG. 21 is a further view of the second embodiment of the intervertebral prosthetic disc;

FIG. 22 is a lateral view of the second embodiment of the intervertebral prosthetic disc;

FIG. 23 is an exploded lateral view of the second embodiment of the intervertebral prosthetic disc;

FIG. 24 is a plan view of a superior half of the second embodiment of the intervertebral prosthetic disc;

FIG. 25 is another plan view of the superior half of the second embodiment of the intervertebral prosthetic disc;

FIG. 26 is a plan view of an inferior half of the second embodiment of the intervertebral prosthetic disc;

FIG. 27 is another plan view of the inferior half of the second embodiment of the intervertebral prosthetic disc;

FIG. 28 is a lateral view of a third embodiment of an intervertebral prosthetic disc;

FIG. 29 is an exploded lateral view of the third embodiment of the intervertebral prosthetic disc;

FIG. 30 is a cross-section view of an exemplary nucleus of the third embodiment of the intervertebral prosthetic disc;

FIG. 31 is an anterior view of the third embodiment of the intervertebral prosthetic disc;

FIG. 32 is a perspective view of a superior component of the third embodiment of the intervertebral prosthetic disc;

FIG. 33 is a perspective view of an inferior component of the third embodiment of the intervertebral prosthetic disc;

FIG. 34 is a lateral view of a fourth embodiment of an intervertebral prosthetic disc;

FIG. 35 is an exploded lateral view of the fourth embodiment of the intervertebral prosthetic disc;

FIG. 36 is a cross-section view of an exemplary nucleus of the fourth embodiment of the intervertebral prosthetic disc;

FIG. 37 is an anterior view of the fourth embodiment of the intervertebral prosthetic disc;

FIG. 38 is a perspective view of a superior component of the fourth embodiment of the intervertebral prosthetic disc;

FIG. 39 is a perspective view of an inferior component of the fourth embodiment of the intervertebral prosthetic disc;

FIG. 40 is a posterior view of a fifth embodiment of an intervertebral prosthetic disc;

FIG. 41 is an exploded posterior view of the fifth embodiment of the intervertebral prosthetic disc;

FIG. 42 is a plan view of a superior half of the fifth embodiment of the intervertebral prosthetic disc;

FIG. 43 is a plan view of an inferior half of the fifth embodiment of the intervertebral prosthetic disc;

FIG. 44 is a perspective view of a sixth embodiment of an intervertebral prosthetic disc;

FIG. 45 is a superior plan view of the sixth embodiment of the intervertebral prosthetic disc;

FIG. 46 is an anterior plan view of the sixth embodiment of the intervertebral prosthetic disc;

FIG. 47 is a cross-section view of the sixth embodiment of the intervertebral prosthetic disc taken along line 43-43 in FIG. 41;

FIG. 48 is a plan view of a nucleus implant installed within an intervertebral disc;

FIG. 49 is a plan view of the nucleus implant within a nucleus delivery device;

FIG. 50 is a plan view of the nucleus implant exiting the nucleus delivery device;

FIG. 51 is a plan view of a nucleus implant installed within an intervertebral disc; and

FIG. 52 and FIG. 53 are plan views of exemplary nucleus implants installed within an intervertebral disc.

### DETAILED DESCRIPTION OF THE DRAWINGS

In a particular embodiment, a prosthetic device, such as a spinal disc implant, includes a component that is adapted to provide a desired mechanical performance of the prosthetic device. For example, a bulk polymeric material of the component of the prosthetic device can be crosslinked to provide a mechanical property. When the component is included in the prosthetic device, the prosthetic device has a desired mechanical performance. In an example, the component can be a nucleus of a spinal disc implant. In another example, the component can include a protrusion formed of crosslinkable bulk polymeric material. The bulk polymeric material of the component can be crosslinked to an extent determined based at least in part on a patient characteristic, a property value, or any combination thereof. Further a portion of the bulk material can be crosslinked to form a component configuration that imparts mechanical performance to the prosthetic device.

In an exemplary embodiment, a method of treating a patient includes determining a patient characteristic associated with the patient, determining a property value based at least in part on the patient characteristic, and determining a crosslinking parameter based at least in part on the property value.

In another exemplary embodiment, a method of forming an implant device component includes determining a configuration of an implant device component and effecting crosslinking in a portion of a bulk polymeric material of the implant device component.

In a further exemplary embodiment, a prosthetic device includes a first component having a depression formed therein and includes a second component having a projection extending therefrom. The projection includes a surface configured to movably engage the depression. A bulk polymeric material of the projection has a crosslinked gradient wherein a fist portion of the bulk polymeric material closer to the surface has a lesser extent of crosslinking than a second portion of the bulk polymeric material further from the surface.

In an additional exemplary embodiment, a prosthetic device includes a first component having a depression formed therein, a second component having a depression formed therein, and a nucleus disposed between the first and second components and configured to movably engage the depressions formed in the first and second components simultaneously. The nucleus is formed of a bulk polymeric material. A first portion of the bulk polymeric material of the nucleus has a greater extent of crosslinking than a second portion of the bulk polymeric material of the nucleus.

In another exemplary embodiment, a prosthetic device includes a component configured to be interposed between two osteal structures. The component is formed of a bulk polymeric material including a first portion of the bulk polymeric material crosslinked to a greater extent than a second portion of the bulk polymeric material.

In a further exemplary embodiment, a kit includes a prosthetic device including a bulk polymeric material. The kit also includes instructions relative to crosslinking the bulk polymeric material.

Description of Relevant Anatomy

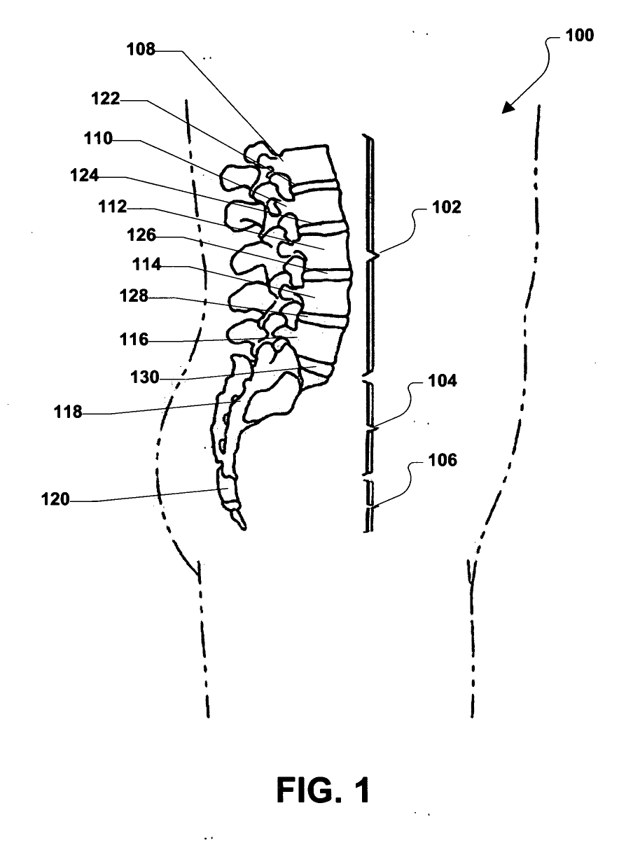

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

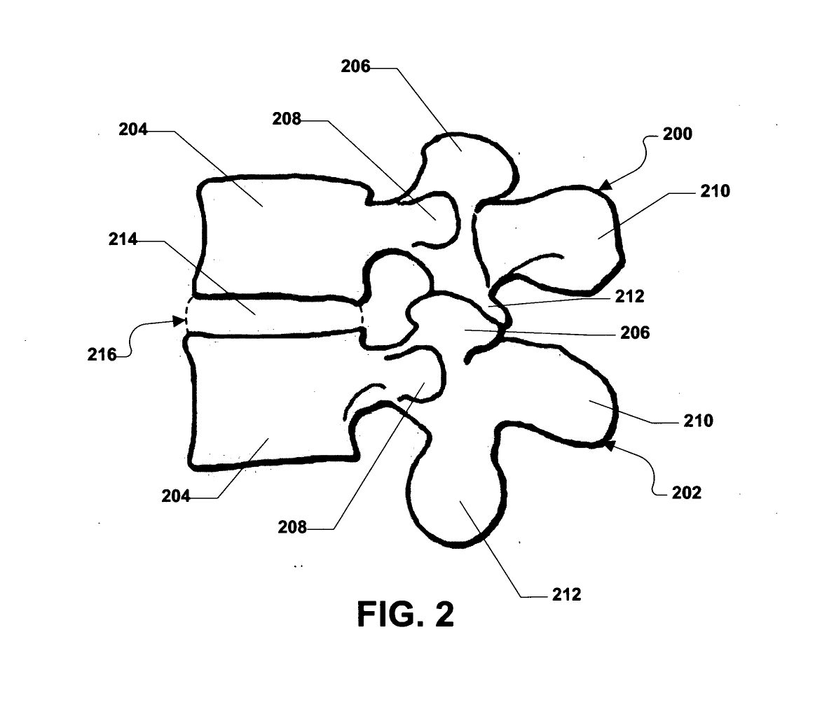

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 214 between the superior vertebra 200 and the inferior vertebra 202.

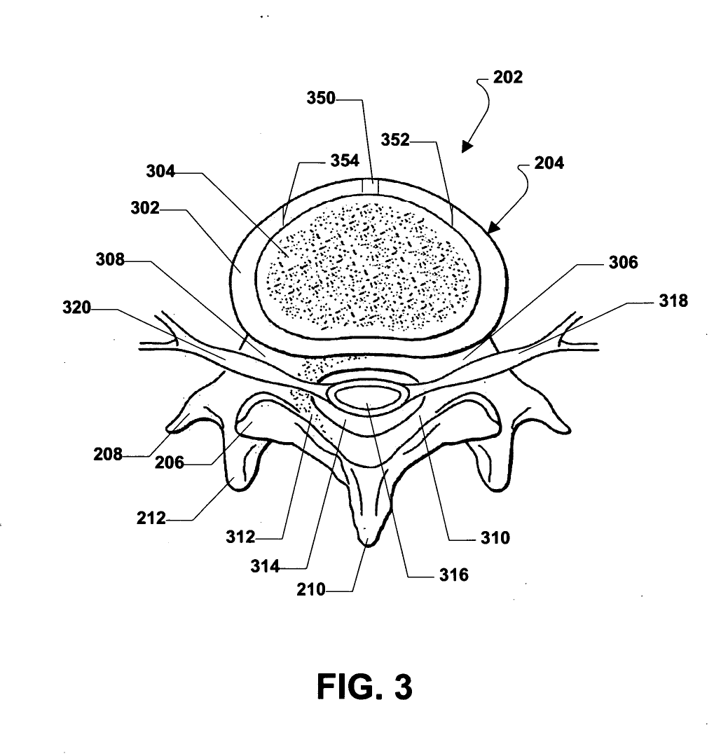

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel-cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

Referring now to FIG. 4, an intervertebral disc is shown and is generally designated 400. The intervertebral disc 400 is made up of two components: the annulus fibrosis 402 and the nucleus pulposus 404. The annulus fibrosis 402 is the outer portion of the intervertebral disc 400; and the annulus fibrosis 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is the inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

Description of a Method for Treating a Patient

In general, a patient may suffer from ailments associated with connections between osteal structures, such as joints between articulated bones or discs between vertebrae. In particular, a patient may suffer from an ailment associated with the degeneration of a disc between superior and inferior vertebrae. Such ailments can be treated using implants. For example, an ailment associated with degeneration of a spinal disc can be treated with an intervertebral prosthetic device.

Based on the characteristics associated with the particular nature of an ailment experienced by a patient, the desired configuration of a prosthetic device can change. For example, performance of the prosthetic device can be a function of mechanical properties of the materials of the prosthetic device. In particular, polymeric prosthetic devices can be crosslinked to alter the mechanical properties of the device. As a result, the polymeric prosthetic device can be tailored based on the characteristics of the patient or the patient's condition.

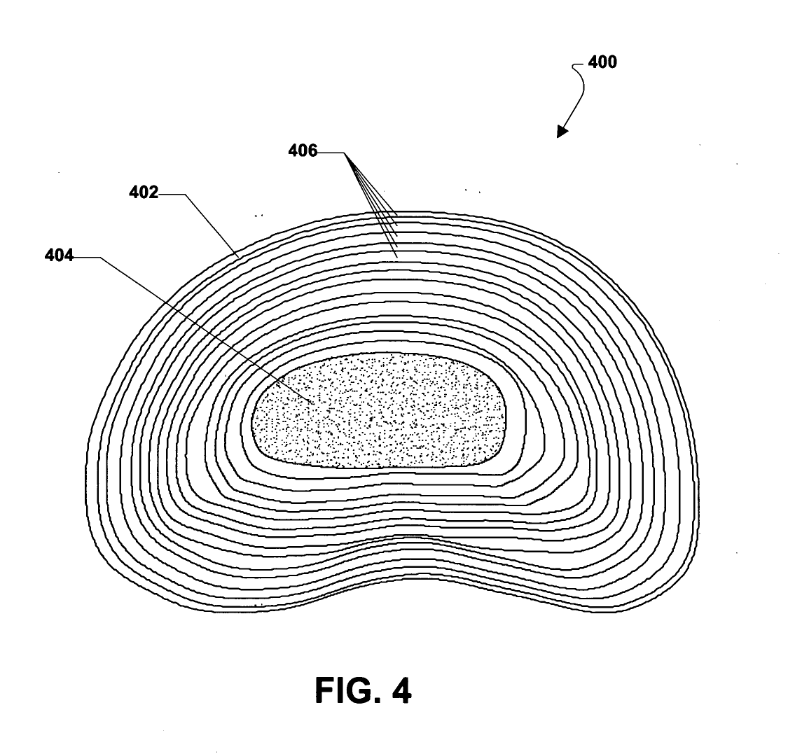

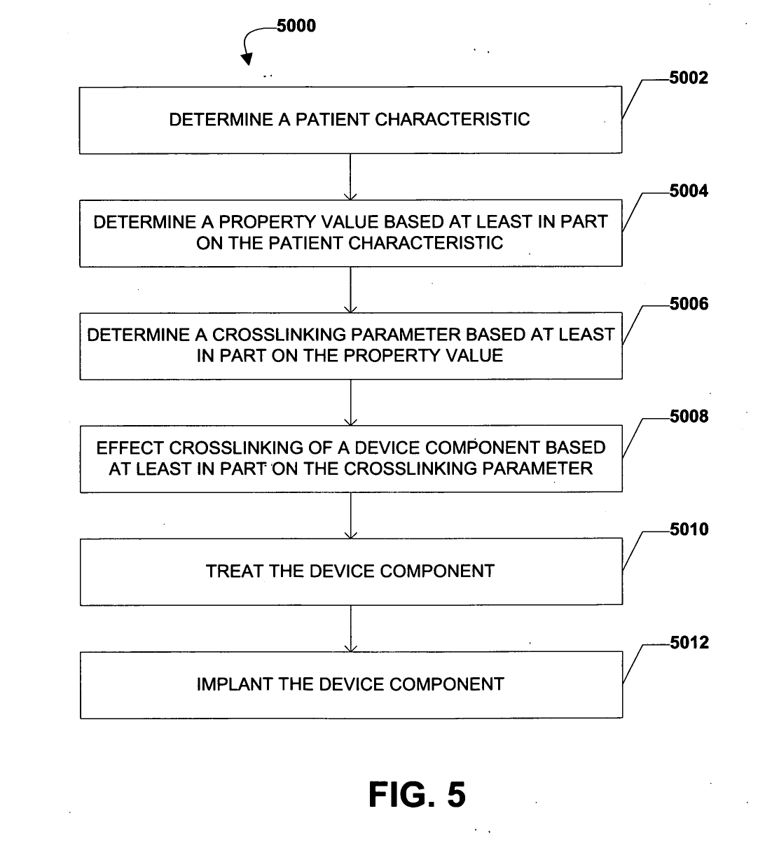

FIG. 5 includes an illustration of an exemplary method 5000 to treat a patient. For example, a patient characteristic associated with a patient or a patient's condition can be determined, as illustrated at 5002. A patient characteristic associated with a patient, for example, can include height, weight, activity level, bone dimensions, or any combination thereof. A patient characteristic associated with a patient's condition can include a grade of degradation or a location of the ailment, such as the region on the spine, a specific intervertebral space, or any combination thereof.

Based at least in part on the patient characteristic, a property value can be determined, as illustrated at 5004. For example, the property value can be associated with the bulk material of a component of a prosthetic device. In general, surface crosslinking can influence surface properties, such as wear resistance, while crosslinking in the bulk material, such as material away from the surface, influences mechanical performance of the prosthetic device. In particular, the property value can relate to compressive modulus, Young's modulus, tensile strength, elongation or strain properties, hardness, or any combination thereof of the bulk material of the component. In a particular example, the prosthetic device can include a nucleus or can include a hemispherical protrusion formed of a crosslinkable polymeric bulk material. The property value, for example, can be a compressive modulus of the bulk material.

Based at least in part on the property value, a crosslinking parameter can be determined, as illustrated at 5006. For example, the crosslinking parameter can be a parameter associated with the crosslinking process. The process for initiating crosslinking of a bulk polymeric material of the component can include a radiative process, a thermal process, a chemical process, or any combination thereof. In an exemplary embodiment, the process is a radiative process, such as a process initiated through exposure of the component to ultraviolet radiation. As such, the crosslinking parameter can be associated with exposure of the component. In a particular example, the crosslinking parameter is a total radiation exposure or a time of exposure to a given intensity or power output of radiation. In another example, the crosslinking parameter can be an amount or concentration of chemical crosslinking agent. In a further example, the crosslinking parameter can include a time of exposure to a temperature or a time of exposure to a radiative heat source. Determining the property value or determining the crosslinking parameter can be automated using software. Alternatively, the determining the property value or determining the crosslinking parameter can be performed using charts, tables, or algorithms. In a further alternative embodiment, a crosslinkable bulk polymeric material may be selected based at least in part on the crosslinking parameter.

Based at least in part on the crosslinking parameter, a portion of the polymeric bulk material of the component can be crosslinked, as illustrated at 5008. For example, crosslinking can be effected by exposure to a radiation source, such as an ultraviolet radiation source, an infrared source, a gamma-radiation source, an e-beam source, or any combination thereof. In another example, crosslinking can be effected by thermal treatment or by chemical treatment. In an example, a portion of the bulk material can be subject to increased temperature, resulting in crosslinking. In general, the crosslinking can result in crosslinking of the bulk material of the component or a portion of the bulk material of the component. When crosslinking is effected in a portion of the bulk material of the component, the bulk material in regions proximate to the portion can be crosslinked to a lesser extent, resulting in a gradient of extent of crosslinking the bulk material. In addition to the crosslinking parameter, a component configuration can be determined. For example, a location within the bulk material at which the crosslinking is to be effected can be determined.

The component optionally can be treated, as illustrated at 5010. For example, the component can be annealed, such as through exposure to elevated temperatures for an extended period. In another example, a surface of the component can be exposed chemical crosslinking agents, resulting in increased crosslinking of the surface. In a further example, the component can be sterilized, such as through exposure to ultraviolet radiation, exposure to gamma radiation, exposure to pressurized steam, or exposure to sterilizing agents, or any combination thereof. Exemplary sterilizing agents include alcohol, anti-microbial agents, or any combination thereof.

The component can be implanted as part of a prosthetic device, as illustrated at 5012. For example, a nucleus of a spinal disc implant can be implanted into the intervertebral space between two vertebrae.

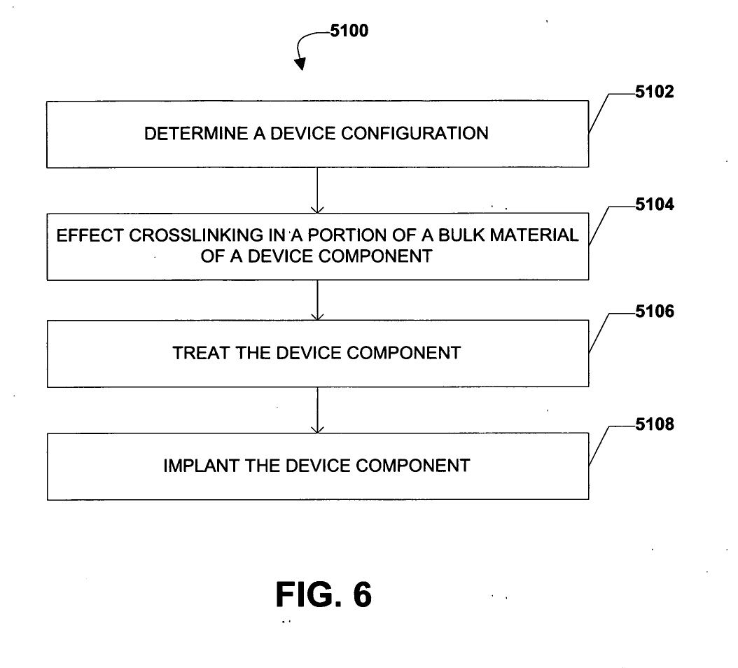

In another example, the performance of a prosthetic device can be influenced by a configuration of components of a prosthetic device. For example, regions of polymeric bulk material of a device component can be selectively crosslinked to influence the performance of prosthetic device. FIG. 6 includes an illustration of an exemplary method 5100 to treat a patient.

In an exemplary embodiment, a device configuration can be determined, as illustrated at 5102. For example, a region of a bulk material to be crosslinked or an extent of crosslinking to be effected at a region can be identified. In an alternative example, a crosslinkable bulk polymeric material may be selected based at least in part on the device configuration. Such configurations can be determined based on patient characteristics or other parameters influencing the selection of device performance characteristics. In a particular embodiment, the device component can be a nucleus of a prosthetic device or a protrusion of the component that imparts performance characteristics to the device based on the material properties of the component. In an exemplary nucleus, the device configuration can include a region of the nucleus to be crosslinked, such as a posterior region, a center region, an anterior region, a left side region, a right side region, or any combination thereof. In an exemplary protrusion of a device component, the device configuration can include an extent of crosslinking within the protrusion.

Based at least in part on the device configuration, crosslinking of the polymeric bulk material of the component can be effected, as illustrated at 5104. For example, the bulk material can be exposed to conditions that result in crosslinking within a region in accordance with the device configuration. For example, a region of a nucleus of a prosthetic device can be exposed to a radiation source while other regions of the nucleus are masked to prevent exposure to the radiation source.

The component optionally can be treated, as illustrated at 5106. For example, the component can be annealed, surface treated, sterilized, or any combination thereof. The component can by implanted, as illustrated at 5108. For example, the component can be included in a prosthetic spinal disc implanted in a patient.

Depending on the application, crosslinking of a component can be effected at time of manufacture, during sterilization, or prior to implantation into a patient. The crosslinking can be effected by equipment located at a medical facility or alternatively, at a remote location or the manufacturers site. In addition, treating the component, such as sterilizing the component can be optionally performed before, during, or after effecting crosslinking. In an exemplary embodiment, crosslinking can be effected at various points during manufacture of the prosthetic disc in order to accommodate various manufacturing parameters, including the desired degree of crosslinking at a portion of the bulk material. Alternatively, crosslinking can be effected post-manufacture, yet prior to implantation (e.g., by surgical staff or the like). In a further particular embodiment, crosslinking can be effected after implantation. Further, crosslinking can be effected at various points between the beginning of manufacture and the end of the implantation procedure. Two or more different crosslinking processes can be performed at various points, as desired, to obtain the desired degree of crosslinking in the desired location(s). In a particular embodiment, crosslinking apparatuses or agents can be provided with all or a portion of the prosthetic disc in kit form for ease of use in the field.

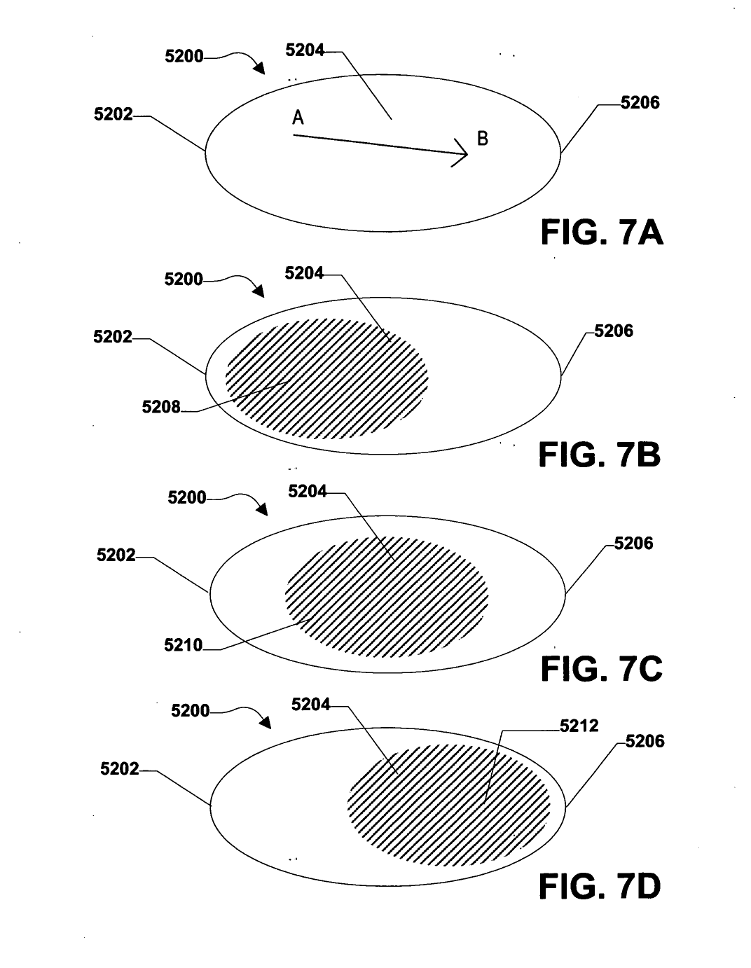

In general, the device configuration can include an extent of crosslinking of the bulk material, a region of crosslinking, or any combination thereof. In an exemplary embodiment, the device component is a nucleus of a prosthetic device. FIGS. 7A, 7B, 7C, and 7D include illustrations of exemplary device configurations. For example, FIG. 7A includes an illustration of a device nucleus 5200 including an anterior portion 5202, a center portion 5204, and a posterior portion 5206. In an exemplary embodiment, a gradient of extent of crosslinking can be formed within the bulk polymeric material of the device nucleus 5200. For example, the bulk polymeric material can have a decreasing extent of crosslinking from point A to point B. As such, the mechanical properties of the bulk polymeric material of the device nucleus 5200 can change along the line extending from point A to point B.

In another exemplary embodiment, crosslinking can be effected at a selected region of a component. As illustrated in FIG. 7B, crosslinking can be effected to a greater extent at an anterior location 5208 than in other locations. Alternatively, crosslinking can be effected at a center location 5210, as illustrated in FIG. 7C, or at a posterior location 5212, as illustrated at FIG. 7D. In another alternative embodiment, crosslinking can be effected at both the posterior and the anterior locations.

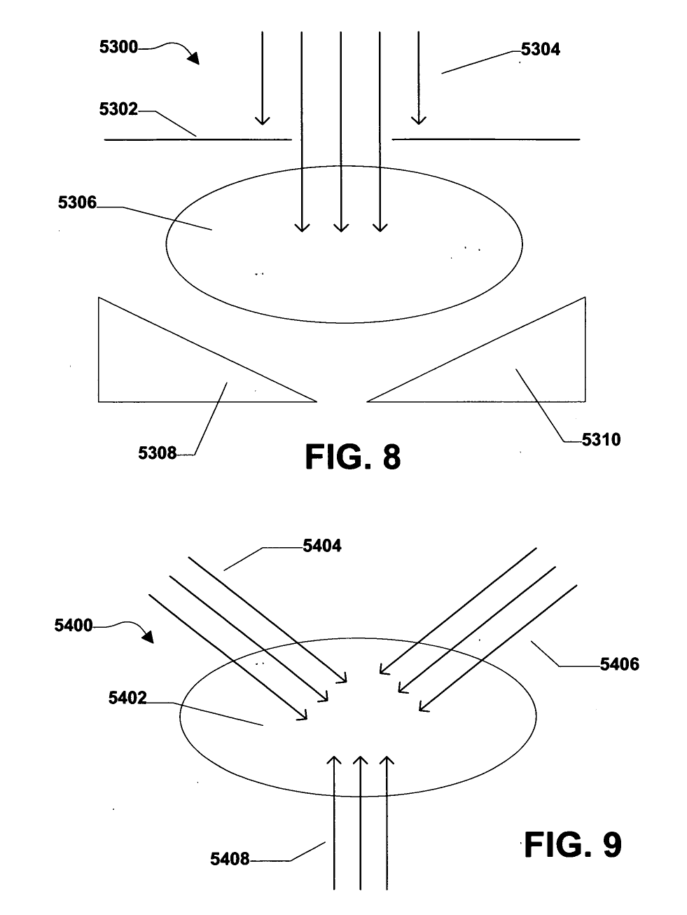

To effect crosslinking in bulk polymeric material in particular regions of the device component, the particular regions can be exposed to radiation, thermal treatment, or chemicals that initiate crosslinking. For example, the particular region can be exposed to irradiation while other portions are shielded from irradiation. For example, FIG. 8 includes an illustration of an exemplary apparatus 5300 for selectively effecting crosslinking in particular regions of a component. A mask 5302 can selectively prevent and allow radiation 5304 from a source to impinge a component 5306. In a particular embodiment, a mask can selectively permit radiation, such as ultraviolet radiation, to pass to the device component 5306. The radiation can effect crosslinking in the regions that are impinged. In addition, a degree of light scattering can effect crosslinking to a lesser extent in regions masked by the mask 5302, forming a crosslinking gradient within the bulk polymeric material of the device component 5306. In addition, the apparatus 5300 can include black bodies 5308 and 5310 to absorb radiation and reduce the amount of reflected radiation effecting crosslinking in masked regions.

FIG. 9 includes an illustration of another exemplary apparatus 5400 for effecting crosslinking in a region of a device component 5402. Radiation 5404, 5406, and 5408 can impinge the component 5402 from different angles. A region of the device can be exposed to the sum of radiation from the three directions while other regions are exposed to less radiation. For example, each of the radiation sources can produce low power radiation that initiates limited crosslinking, while the sum of the radiation from the radiation sources initiates increased crosslinking. Regions exposed to one or fewer of the sources can crosslink to a small extent or can not crosslink. A region exposed to each of the radiation sources can crosslink to a high extent. As such, the bulk material of a region of the component can have high crosslinking relative to the bulk material in other regions of the component.

In an exemplary embodiment, an apparatus to effect crosslinking of a portion of a component of a prosthetic device may be manufactured and sold or leased to a medical facility or prosthetics lab. In addition, a kit may be provided that includes a prosthetic device including crosslinkable bulk polymeric material and that includes instructions relating to crosslinking the bulk polymeric material, such as a portion of the bulk polymeric material. Such instructions may include a chart, a table, an algorithm, or software to determine a crosslinking parameter or a device configuration based at least in part on a patient characteristic; a property value, or any combination thereof.

Description of the Bulk Polymeric Materials for Use in Prosthetic Devices

In general, components of the prosthetic device are formed of biocompatible materials. For example, components can be formed of metallic material or of polymeric material. An exemplary metallic material includes titanium, titanium alloy, tantalum, tantalum alloy, zirconium, zirconium alloy, stainless steel, cobalt, cobalt containing alloy, chromium containing alloy, indium tin oxide, silicon, magnesium containing alloy, or any combination thereof.

The bulk polymer materials of components of the prosthetic device are generally biocompatible. An example bulk polymeric material can include a polyurethane material, a polyolefin material, a polystyrene, a polyurea, a polyamide, a polyaryletherketone (PAEK) material, a silicone material, a hydrogel material, or any alloy, blend or copolymer thereof. An exemplary polyolefin material can include polypropylene, polyethylene, halogenated polyolefin, fluoropolyolefin, polybutadiene, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof.

In particular, portions of the prosthetic device can be formed of crosslinkable bulk polymeric materials. For example, a bulk polymeric material can include crosslinkable polymer that is crosslinkable without additives. In another example, additives can be blended into the bulk polymeric material to initiate crosslinking or to form crosslinks. The bulk polymeric material can be crosslinkable through processes such as exposure to radiation, thermal exposure, or exposure to chemical agents. An exemplary radiation includes ultraviolet radiation, gamma-radiation, infrared radiation, e-beam particle radiation, or any combination thereof.

In an exemplary embodiment, the bulk polymeric material is crosslinkable using radiation. The bulk polymeric material can include a photoinitiator or a photosensitizer. In another exemplary embodiment, the bulk polymeric material is thermally crosslinkable and includes a heat activated catalyst. Further, the bulk polymeric material can include a crosslinking agent, which can act to form crosslinks between polymer chains.

For example, for polyurethane materials, a suitable chemical crosslinking agent can include low molecular weight polyols or polyamines. An example of such a suitable chemical crosslinking agent can include trimethylolpropane, pentaerythritol, ISONOL® 93 curative from Dow Chemical Co., trimethylolethane, triethanolamine, Jeffamines, 1,4-butanediamine, xylene diamine, diethylenetriamine, methylene dianiline, diethanolamine, or any combination thereof.

For silicone materials, a suitable chemical crosslinking agent can include tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy) propyltriethoxysilane, 1,2-bis(trimethoxysilyl)ethane, 1,2-bis(triethoxysilyl)ethane, hexaethoxydisiloxane, or any combination thereof.

Additionally, for polyolefin materials, a suitable chemical crosslinking agent can include an isocyanate, a polyol, a polyamine, or any combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or any combination thereof. The polyol can include polyether polyol, hydroxy-terminated polybutadiene, polyester polyol, polycaprolactone polyol, polycarbonate polyol, or any combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or any combination thereof.

In another embodiment, the chemical crosslinking agent is a polyol curing agent. The polyol curing agent can include ethylene glycol; diethylene glycol; polyethylene glycol; propylene glycol; polypropylene glycol; lower molecular weight polytetramethylene ether glycol; 1,3-bis(2-hydroxyethoxy) benzene; 1,3-bis-[2-(2-hydroxyethoxy)ethoxy]benzene; 1,3-bis-{2-[2-(2-hydroxyethoxy) ethoxy]ethoxy}benzene; 1,4-butanediol; 1,5-pentanediol; 1,6-hexanediol; resorcinol-di-(β-hydroxyethyl) ether; hydroquinone-di-(β-hydroxyethyl) ether; trimethylol propane, and any mixtures thereof.

In a particular embodiment, the amount of crosslinking can vary depending on the type of material to be crosslinked, the time of exposure of the material to the crosslinking agent, the type of catalyst, etc. Also, in a particular embodiment, the component can be crosslinked at a depth of greater than about three millimeters (3 mm). In this manner, the bulk polymeric material underlying a surface can exhibit the desired material properties whether or not the surface is crosslinked. In a particular embodiment, the surface remains uncrosslinked or is crosslinked to an extent less than a particular portion of the bulk material.

Accordingly, the hardness of a crosslinked portion can be greater than the hardness of other portions. Further, the Young's modulus or compressive modulus of a crosslinked portion can be greater than the Young's modulus or compressive modulus of another portion. Also, the toughness of the crosslinked portion can be greater than the toughness of other portions of the bulk polymeric material. In a particular embodiment, the compressive modulus of the crosslinked portion can be at least about 5% greater than the compressive modulus of other portions of the bulk material. For example, the compressive modulus of the crosslinked portion can be at least about 10% greater, such as at least about 20% greater or even at least about 50% greater, than the compressive modulus of other portions of the bulk material. In an exemplary embodiment, the compressive modulus is between about 1.0 MPa to about 20 GPa, such as between about 5 MPa to about 5 GPa or between about 0.5 GPa to about 4 GPa.

Description of a First Embodiment of an Intervertebral Prosthetic Disc

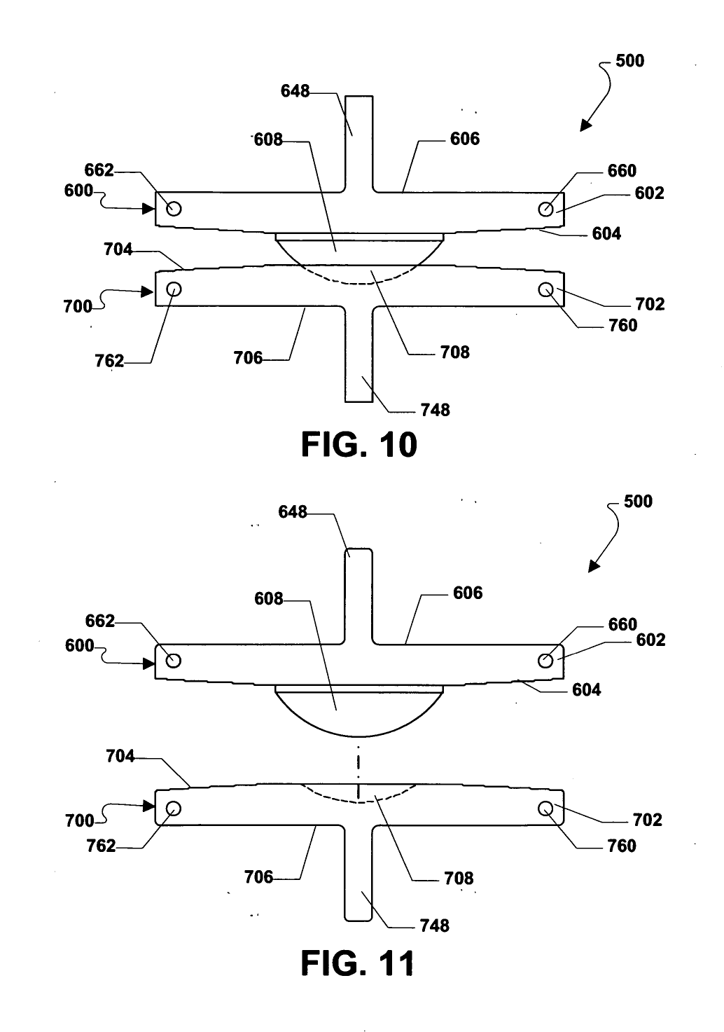

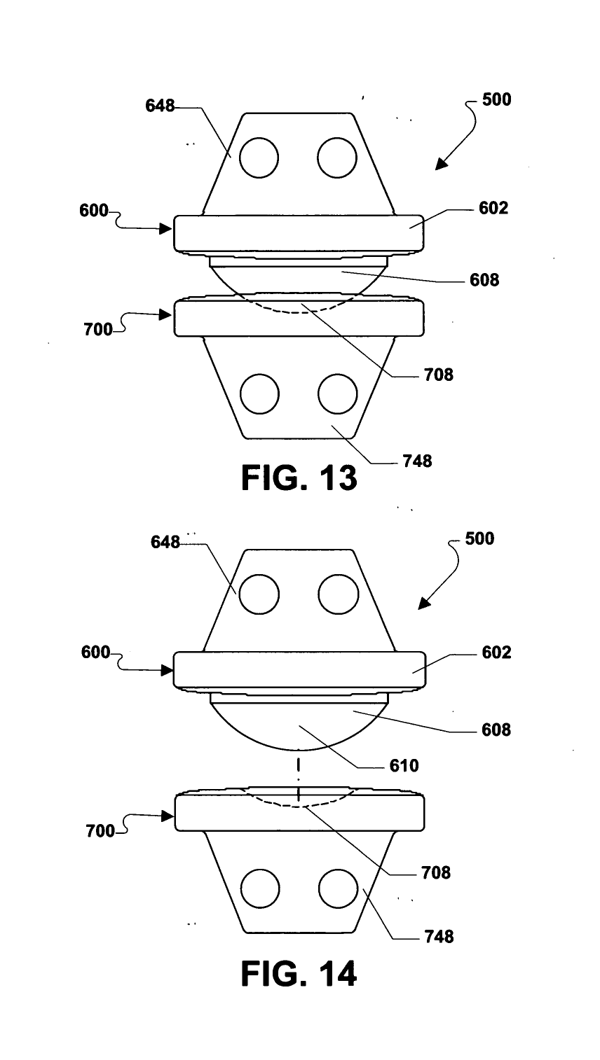

Referring to FIGS. 10 through 18, a first embodiment of an intervertebral prosthetic disc is shown and is generally designated 500. As illustrated, the intervertebral prosthetic disc 500 can include a superior component 600 and an inferior component 700. In a particular embodiment, the components 600, 700 can be made from one or more biocompatible materials. For example, the biocompatible materials can be one or more polymer materials.

In a particular embodiment, the superior component 600 can include a superior support plate 602 that has a superior articular surface 604 and a superior bearing surface 606. In a particular embodiment, the superior articular surface 604 can be generally curved and the superior bearing surface 606 can be substantially flat. In an alternative embodiment, the superior articular surface 604 can be substantially flat and at least a portion of the superior bearing surface 606 can be generally curved.

As illustrated in FIG. 10 through FIG. 14, a projection 608 extends from the superior articular surface 604 of the superior support plate 602. In a particular embodiment, the projection 608 has a hemi-spherical shape. Alternatively, the projection 608 can have an elliptical shape, a cylindrical shape, or other arcuate shape. The projection 608 can be formed of crosslinkable polymeric material.

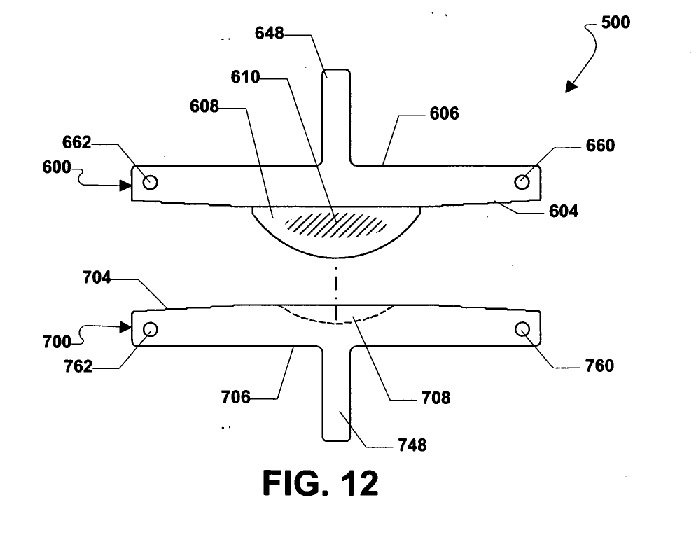

Referring to FIG. 12, the projection 608 can include an interior crosslinked region 610. In a particular embodiment, the interior crosslinked region 610 within the bulk polymeric material forming the projection 608 is crosslinked to a greater extent than other portions of the projection 608. In a particular example, the interior crosslinked region 610 is proximate to a center of the projection 608 and is crosslinked to a greater extent that other regions radially distant from the center of the projection. As such, the extent of crosslinking can decrease with distance from the center of the projection 608.

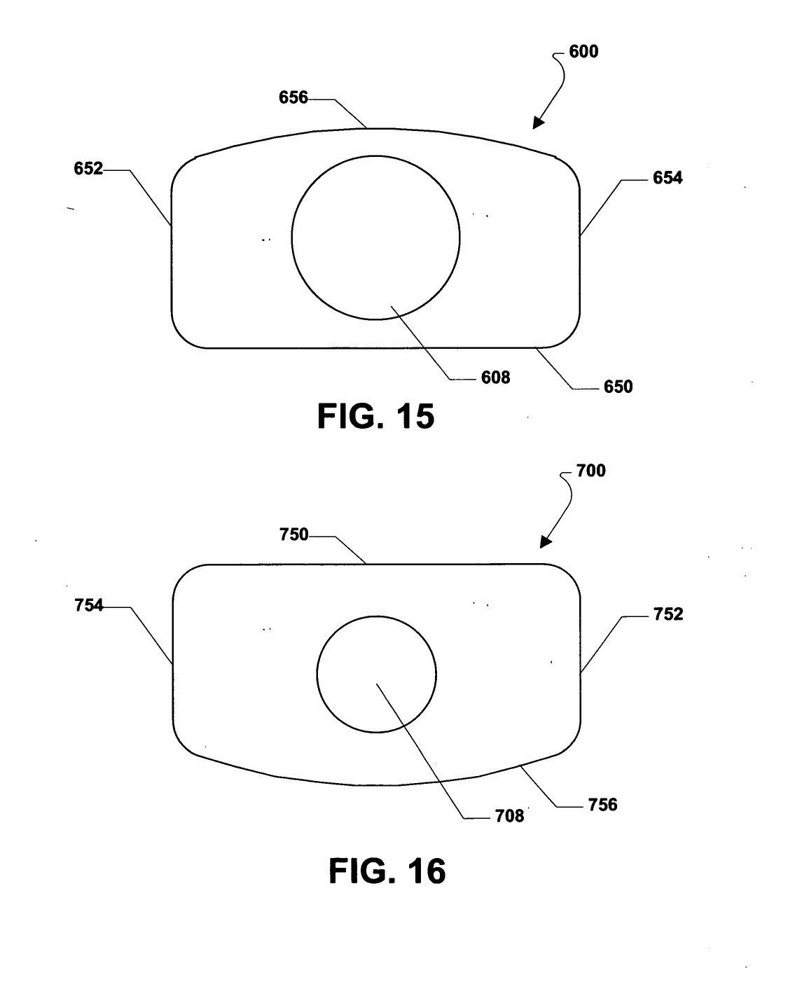

As illustrated in FIG. 15, the superior component 600 can be generally rectangular in shape. For example, the superior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the superior component 600 is wider through the middle than along the lateral sides 652, 654. Further, in a particular embodiment, the lateral sides 652, 654 are substantially the same length.

FIG. 10 through FIG. 12 show that the superior component 600 can include a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 500 shown in FIG. 10 through FIG. 18.

In a particular embodiment, the inferior component 700 can include an inferior support plate 702 that has an inferior articular surface 704 and an inferior bearing surface 706. In a particular embodiment, the inferior articular surface 704 can be generally curved and the inferior bearing surface 706 can be substantially flat. In an alternative embodiment, the inferior articular surface 704 can be substantially flat and at least a portion of the inferior bearing surface 706 can be generally curved.

As illustrated in FIG. 10 through FIG. 14, a depression 708 extends into the inferior articular surface 704 of the inferior support plate 702. In a particular embodiment, the depression 708 is sized and shaped to receive the projection 608 of the superior component 600. For example, the depression 708 can have a hemispherical shape. Alternatively, the depression 708 can have an elliptical shape, a cylindrical shape, or other arcuate shape.

FIG. 10 through FIG. 14 indicate that the superior component 600 can include a superior keel 648 that extends from superior bearing surface 606 and the inferior component 700 can include an inferior keel 748 that extends from inferior bearing surface 706. During installation, described below, the superior keel 648 and the inferior keel 748 can at least partially engage a keel groove that can be established within a cortical rim of a vertebra, e.g., the keel groove 350 shown in FIG. 3. Further, the superior keel 648 or the inferior keel 748 can be coated with a bone-growth promoting substance, e.g., a hydroxyapatite coating formed of calcium phosphate. Additionally, the superior bearing surface 606 or the inferior bearing surface 706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

In a particular embodiment, as shown in FIG. 16, the inferior component 700 can be shaped to match the shape of the superior component 600, shown in FIG. 15. Further, the inferior component 700 can be generally rectangular in shape. For example, the inferior component 700 can have a substantially straight posterior side 750. A first straight lateral side 752 and a second substantially straight lateral side 754 can extend substantially perpendicular from the posterior side 750 to an anterior side 756. In a particular embodiment, the anterior side 756 can curve outward such that the inferior component 700 is wider through the middle than along the lateral sides 752, 754. Further, in a particular embodiment, the lateral sides 752, 754 are substantially the same length.

FIG. 10 through FIG. 12 show that the inferior component 700 can include a first implant inserter engagement hole 760 and a second implant inserter engagement hole 762. In a particular embodiment, the implant inserter engagement holes 760, 762 are configured to receive respective dowels, or pins, that extend from an implant inserter (not shown) that can be used to facilitate the proper installation of an intervertebral prosthetic disc, e.g., the intervertebral prosthetic disc 500 shown in FIG. 10 through FIG. 16.

In a particular embodiment, the overall height of the intervertebral prosthetic device 500 can be in a range from fourteen millimeters to forty-six millimeters (14-46 mm). Further, the installed height of the intervertebral prosthetic device 500 can be in a range from eight millimeters to sixteen millimeters (8-16 mm). In a particular embodiment, the installed height can be substantially equivalent to the distance between an inferior vertebra and a superior vertebra when the intervertebral prosthetic device 500 is installed therebetween.

In a particular embodiment, the length of the intervertebral prosthetic device 500, e.g., along a longitudinal axis, can be in a range from thirty millimeters to forty millimeters (30-40 mm). Additionally, the width of the intervertebral prosthetic device 500, e.g., along a lateral axis, can be in a range from twenty-five millimeters to forty millimeters (25-40 mm). Moreover, in a particular embodiment, each keel 648, 748 can have a height in a range from three millimeters to fifteen millimeters (3-15 mm).

Installation of the First Embodiment within an Intervertebral Space

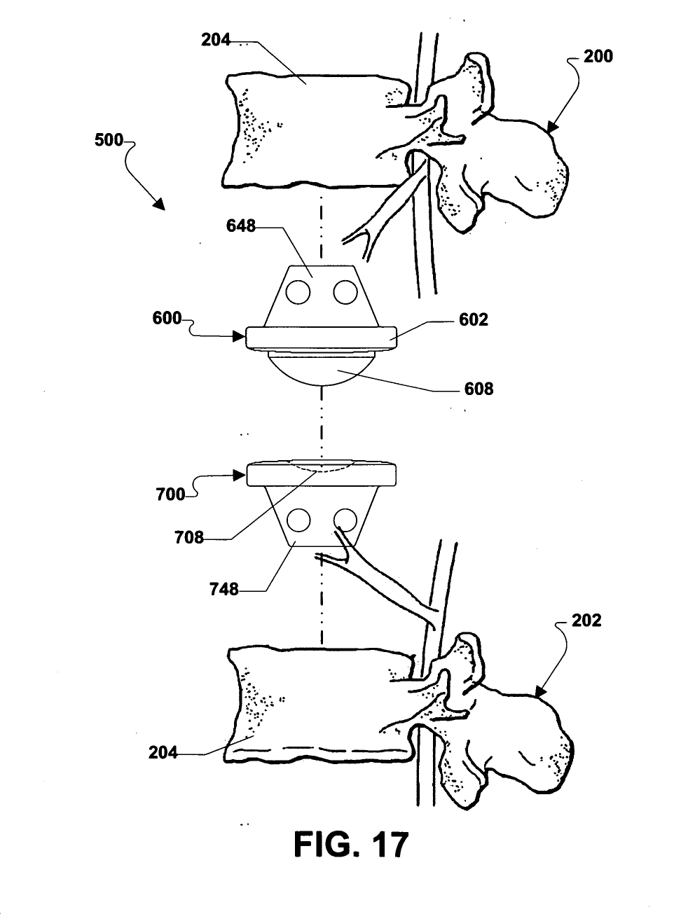

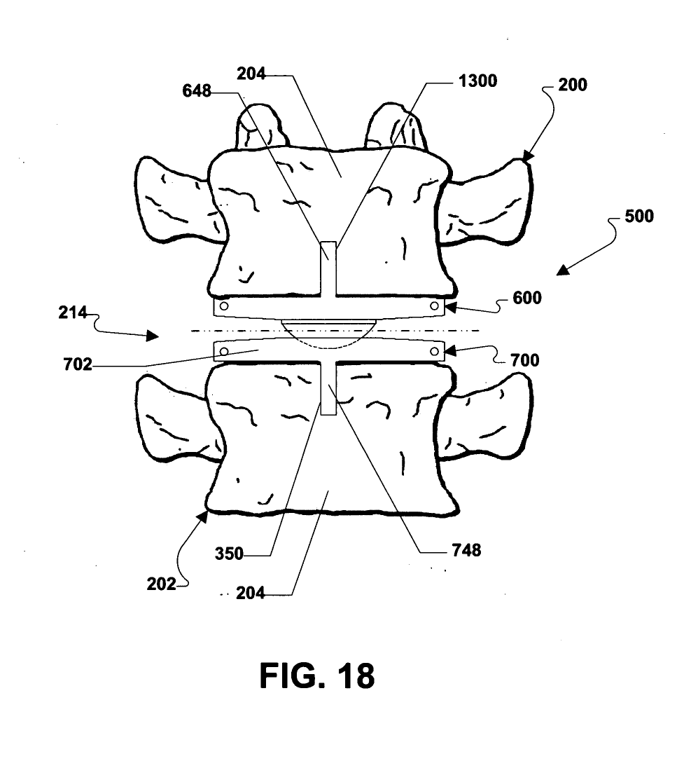

Referring to FIG. 17 and FIG. 18, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 500 described in conjunction with FIG. 10 through FIG. 16. Alternatively, the intervertebral prosthetic disc can be an intervertebral prosthetic disc according to any of the embodiments disclosed herein.

As shown in FIG. 17 and FIG. 18, the intervertebral prosthetic disc 500 is installed within the intervertebral space 214 that can be established between the superior vertebra 200 and the inferior vertebra 202 by removing vertebral disc material (not shown). FIG. 18 shows that the superior keel 648 of the superior component 600 can at least partially engage the cancellous bone and cortical rim of the superior vertebra 200. Further, as shown in FIG. 18, the superior keel 648 of the superior component 600 can at least partially engage a superior keel groove 1300 that can be established within the vertebral body 204 of the superior vertebra 202. In a particular embodiment, the vertebral body 204 can be further cut to allow the superior support plate 602 of the superior component 600 to be at least partially recessed into the vertebral body 204 of the superior vertebra 200.

Also, as shown in FIG. 18, the inferior keel 748 of the inferior component 700 can at least partially engage the cancellous bone and cortical rim of the inferior vertebra 202. Further, as shown in FIG. 18, the inferior keel 748 of the inferior component 700 can at least partially engage the inferior keel groove 350, previously introduced and described in conjunction with FIG. 3, which can be established within the vertebral body 204 of the inferior vertebra 202. In a particular embodiment, the vertebral body 204 can be further cut to allow the inferior support plate 702 of the inferior component 700 to be at least partially recessed into the vertebral body 204 of the inferior vertebra 200.

It is to be appreciated that when the intervertebral prosthetic disc 500 is installed between the superior vertebra 200 and the inferior vertebra 202, the intervertebral prosthetic disc 500 allows relative motion between the superior vertebra 200 and the inferior vertebra 202. Specifically, the configuration of the superior component 600 and the inferior component 700 allows the superior component 600 to rotate with respect to the inferior component 700. As such, the superior vertebra 200 can rotate with respect to the inferior vertebra 202. In a particular embodiment, the intervertebral prosthetic disc 500 can allow angular movement in any radial direction relative to the intervertebral prosthetic disc 500.

Further, as depicted in FIGS. 16 through 18, the inferior component 700 can be placed on the inferior vertebra 202 so that the center of rotation of the inferior component 700 is substantially aligned with the center of rotation of the inferior vertebra 202. Similarly, the superior component 600 can be placed relative to the superior vertebra 200 so that the center of rotation of the superior component 600 is substantially aligned with the center of rotation of the superior vertebra 200. Accordingly, when the vertebral disc, between the inferior vertebra 202 and the superior vertebra 200, is removed and replaced with the intervertebral prosthetic disc 500 the relative motion of the vertebrae 200, 202 provided by the vertebral disc is substantially replicated.

Description of a Second Embodiment of an Intervertebral Prosthetic Disc

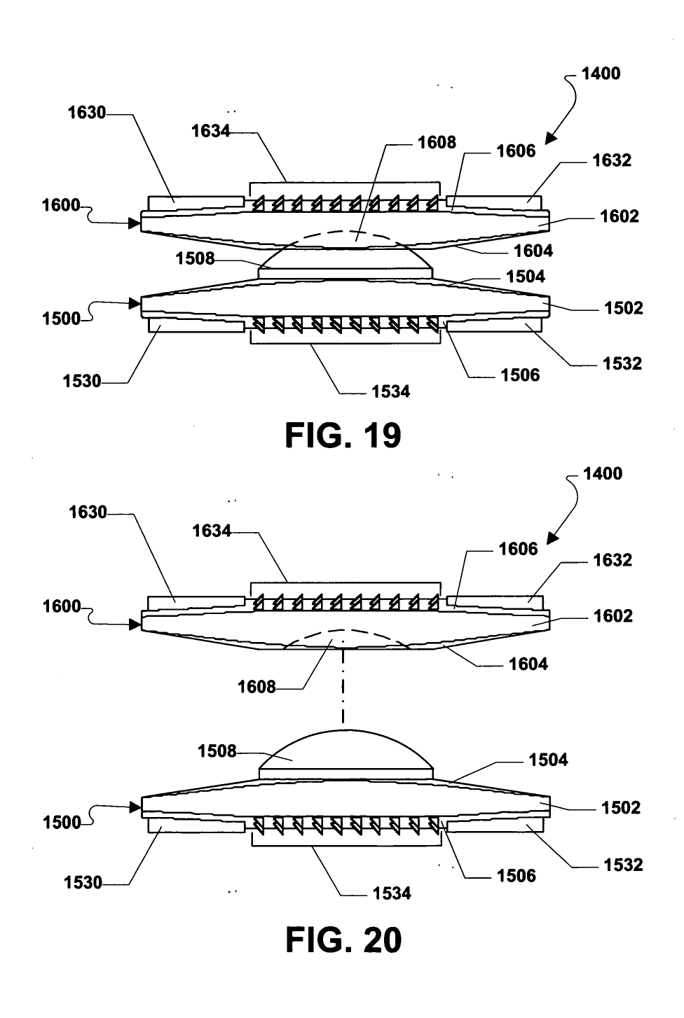

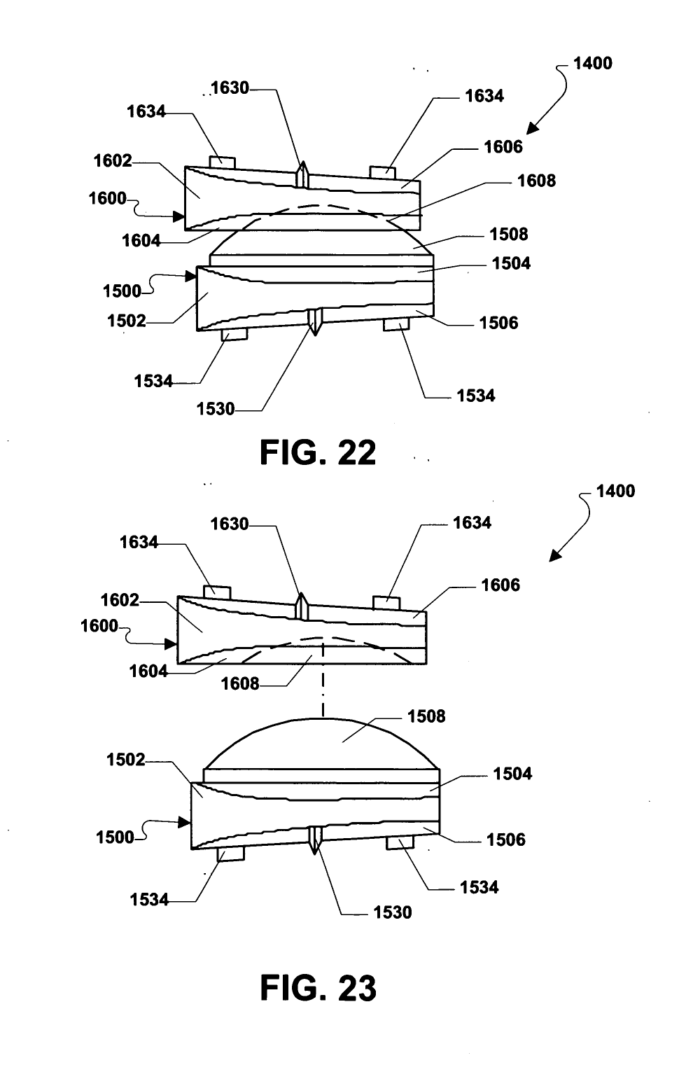

Referring to FIGS. 19 through 27, a second embodiment of an intervertebral prosthetic disc is shown and is generally designated 1400. As illustrated, the intervertebral prosthetic disc 1400 can include an inferior component 1500 and a superior component 1600. In a particular embodiment, the components 1500, 1600 can be made from one or more biocompatible materials. For example, the biocompatible materials can be one or more polymer materials.

In a particular embodiment, the inferior component 1500 can include an inferior support plate 1502 that has an inferior articular surface 1504 and an inferior bearing surface 1506. In a particular embodiment, the inferior articular surface 1504 can be generally rounded and the inferior bearing surface 1506 can be generally flat.

As illustrated in FIG. 19 through FIG. 27, a projection 1508 extends from the inferior articular surface 1504 of the inferior support plate 1502. In a particular embodiment, the projection 1508 has a hemispherical shape. Alternatively, the projection 1508 can have an elliptical shape, a cylindrical shape, or other arcuate shape.

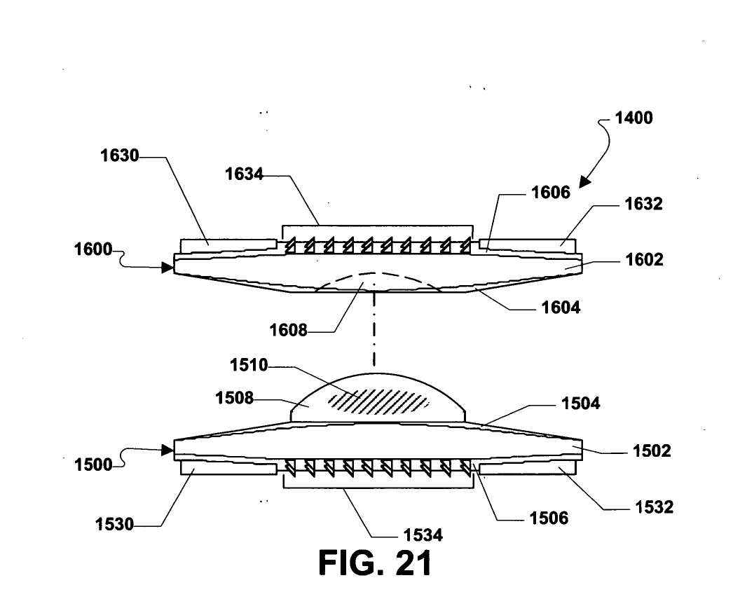

Referring to FIG. 21, the projection 1508 can include a bulk polymeric material including a crosslinked portion 1510. For example, the crosslinked portion 1510 can be crosslinked to an extent that provides desired mechanical response. Such a mechanical response can be determined based on patient characteristics.

Accordingly, the hardness of the crosslinked portion 1510 can be greater than the hardness of other portions of the projection 1508. Further, the Young's modulus or the compressive modulus of the crosslinked portion 1510 can be greater than the Young's modulus or the compressive modulus of other portions. Also, the toughness of the crosslinked portion 1510 can be greater than the toughness of other portions.

FIG. 19 through FIG. 23 and FIG. 25 also show that the inferior component 1500 can include a first inferior keel 1530, a second inferior keel 1532, and a plurality of inferior teeth 1534 that extend from the inferior bearing surface 1506. As shown, in a particular embodiment, the inferior keels 1530, 1532 and the inferior teeth 1534 are generally saw-tooth, or triangle, shaped. Further, the inferior keels 1530, 1532 and the inferior teeth 1534 are designed to engage cancellous bone, cortical bone, or a combination thereof of an inferior vertebra. Additionally, the inferior teeth 1534 can prevent the inferior component 1500 from moving with respect to an inferior vertebra after the intervertebral prosthetic disc 1400 is installed within the intervertebral space between the inferior vertebra and the superior vertebra. In a particular embodiment, the inferior teeth 1534 can include other projections such as spikes, pins, blades, or a combination thereof that have any cross-sectional geometry.

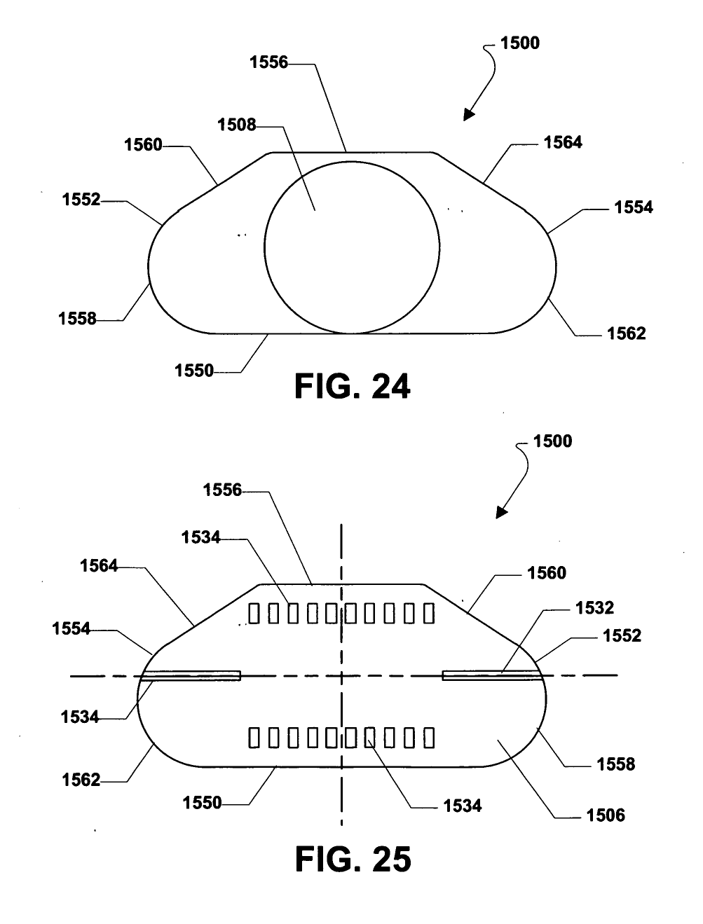

As illustrated in FIG. 24 and FIG. 25, the inferior component 1500 can be generally shaped to match the general shape of the vertebral body of a vertebra. For example, the inferior component 1500 can have a general trapezoid shape and the inferior component 1500 can include a posterior side 1550. A first lateral side 1552 and a second lateral side 1554 can extend from the posterior side 1550 to an anterior side 1556. In a particular embodiment, the first lateral side 1552 can include a curved portion 1558 and a straight portion 1560 that extends at an angle toward the anterior side 1556. Further, the second lateral side 1554 can also include a curved portion 1562 and a straight portion 1564 that extends at an angle toward the anterior side 1556.

As shown in FIG. 24 and FIG. 25, the anterior side 1556 of the inferior component 1500 can be relatively shorter than the posterior side 1550 of the inferior component 1500. Further, in a particular embodiment, the anterior side 1556 is substantially parallel to the posterior side 1550. As indicated in FIG. 19, the projection 1508 can be situated relative to the inferior articular surface 1504 such that the perimeter of the projection 1508 is tangential to the posterior side 1550 of the inferior component 1500. In alternative embodiments (not shown), the projection 1508 can be situated relative to the inferior articular surface 1504 such that the perimeter of the projection 1508 is tangential to the anterior side 1556 of the inferior component 1500 or tangential to both the anterior side 1556 and the posterior side 1550.

In a particular embodiment, the superior component 1600 can include a superior support plate 1602 that has a superior articular surface 1604 and a superior bearing surface 1606. In a particular embodiment, the superior articular surface 1604 can be generally rounded and the superior bearing surface 1606 can be generally flat.

As illustrated in FIG. 19 through FIG. 27, a depression 1608 extends into the superior articular surface 1604 of the superior support plate 1602. In a particular embodiment, the depression 1608 has a hemi-spherical shape. Alternatively, the depression 1608 can have an elliptical shape, a cylindrical shape, or other arcuate shape.

FIG. 19 through FIG. 23 and FIG. 27 also show that the superior component 1600 can include a first superior keel 1630, a second superior keel 1632, and a plurality of superior teeth 1634 that extend from the superior bearing surface 1606. As shown, in a particular embodiment, the superior keels 1630, 1632 and the superior teeth 1634 are generally saw-tooth, or triangle, shaped. Further, the superior keels 1630, 1632 and the superior teeth 1634 are designed to engage cancellous bone, cortical bone, or a combination thereof, of a superior vertebra. Additionally, the superior teeth 1634 can prevent the superior component 1600 from moving with respect to a superior vertebra after the intervertebral prosthetic disc 1400 is installed within the intervertebral space between the inferior vertebra and the superior vertebra. In a particular embodiment, the superior teeth 1634 can include other depressions such as spikes, pins, blades, or a combination thereof that have any cross-sectional geometry.

In a particular embodiment, the superior component 1600 can be shaped to match the shape of the inferior component 1500 shown in FIG. 24 and FIG. 25. Further, the superior component 1600 can be shaped to match the general shape of a vertebral body of a vertebra. For example, the superior component 1600 can have a general trapezoid shape and the superior component 1600 can include a posterior side 1650. A first lateral side 1652 and a second lateral side 1654 can extend from the posterior side 1650 to an anterior side 1656. In a particular embodiment, the first lateral side 1652 can include a curved portion 1658 and a straight portion 1660 that extends at an angle toward the anterior side 1656. Further, the second lateral side 1654 can also include a curved portion 1662 and a straight portion 1664 that extends at an angle toward the anterior side 1656.

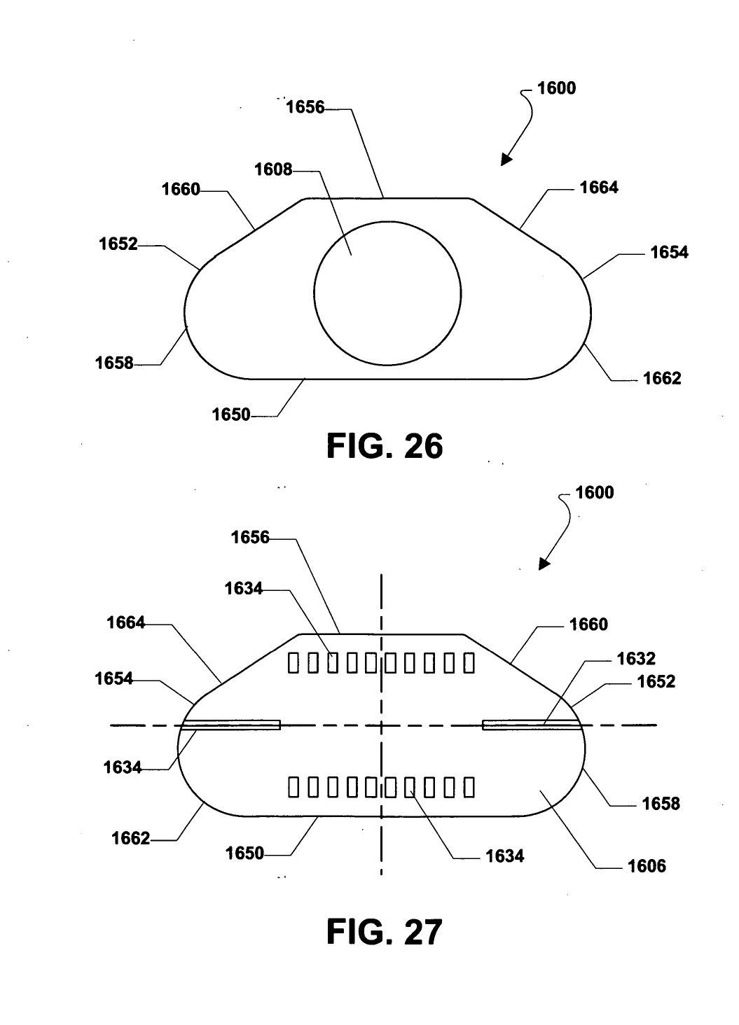

As shown in FIG. 26 and FIG. 27, the anterior side 1656 of the superior component 1600 can be relatively shorter than the posterior side 1650 of the superior component 1600. Further, in a particular embodiment, the anterior side 1656 is substantially parallel to the posterior side 1650.

In a particular embodiment, the overall height of the intervertebral prosthetic device 1400 can be in a range from six millimeters to twenty-two millimeters (6-22 mm). Further, the installed height of the intervertebral prosthetic device 1400 can be in a range from four millimeters to sixteen millimeters (4-16 mm). In a particular embodiment, the installed height can be substantially equivalent to the distance between an inferior vertebra and a superior vertebra when the intervertebral prosthetic device 1400 is installed therebetween.

In a particular embodiment, the length of the intervertebral prosthetic device 1400, e.g., along a longitudinal axis, can be in a range from thirty-three millimeters to fifty millimeters (33-50 mm). Additionally, the width of the intervertebral prosthetic device 1400, e.g., along a lateral axis, can be in a range from eighteen millimeters to twenty-nine millimeters (18-29 mm).

In a particular embodiment, the intervertebral prosthetic disc 1400 can be considered to be “low profile.” The low profile the intervertebral prosthetic device 1400 can allow the intervertebral prosthetic device 1400 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 1518, 1618 can be oriented to engage in a direction substantially opposite the direction of insertion of the prosthetic disc into the intervertebral space.

Further, the intervertebral prosthetic disc 1400 can have a general “bullet” shape as shown in the posterior plan view, described herein. The bullet shape of the intervertebral prosthetic disc 1400 can further allow the intervertebral prosthetic disc 1400 to be inserted through the patient's psoas muscle while minimizing risk to the patient's spinal cord and sympathetic chain.

Description of a Third Embodiment of an Intervertebral Prosthetic Disc

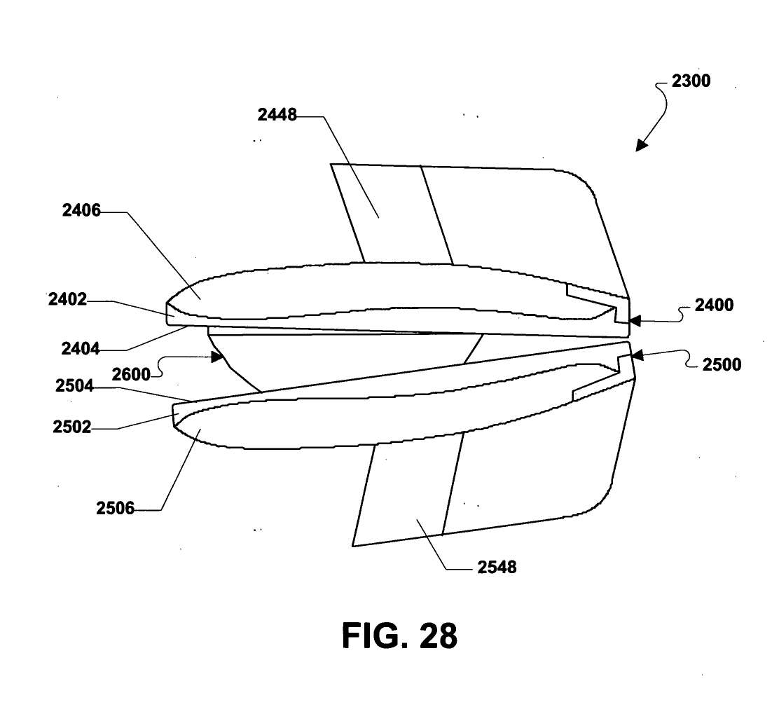

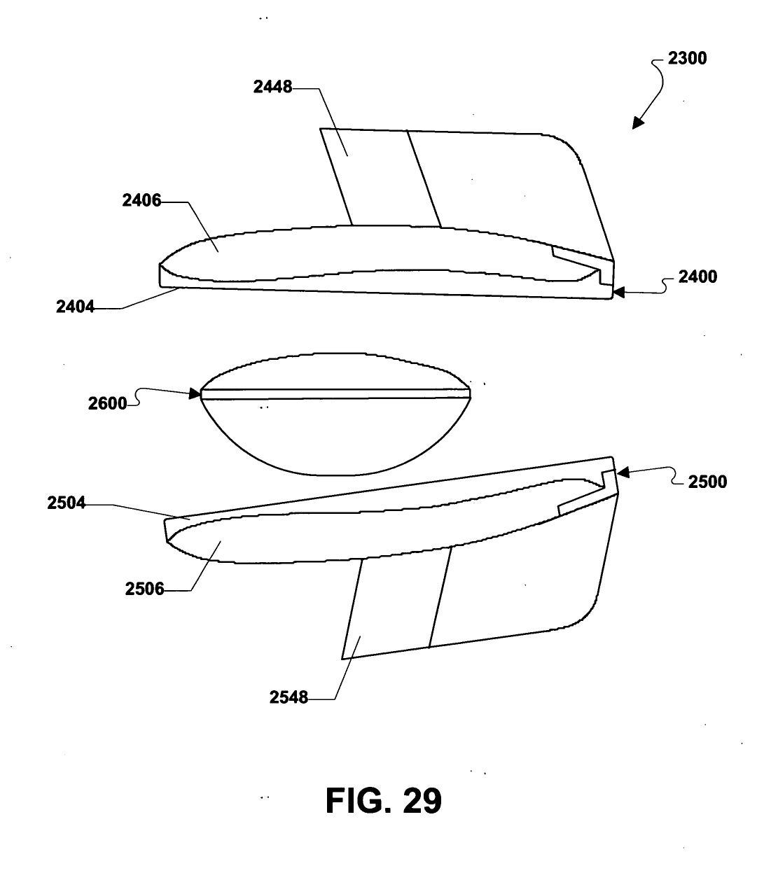

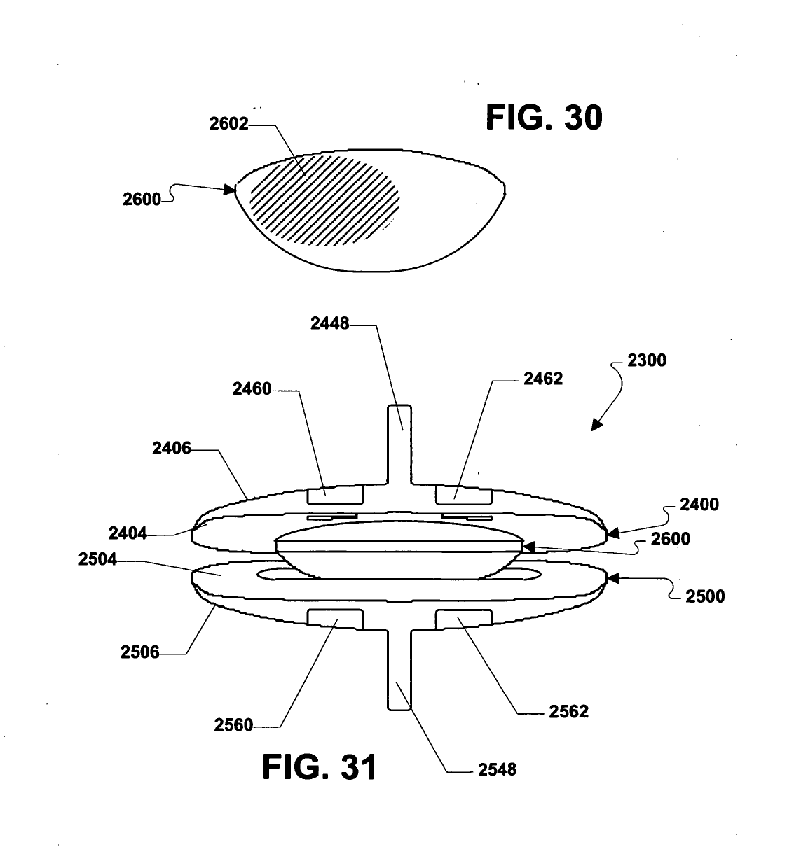

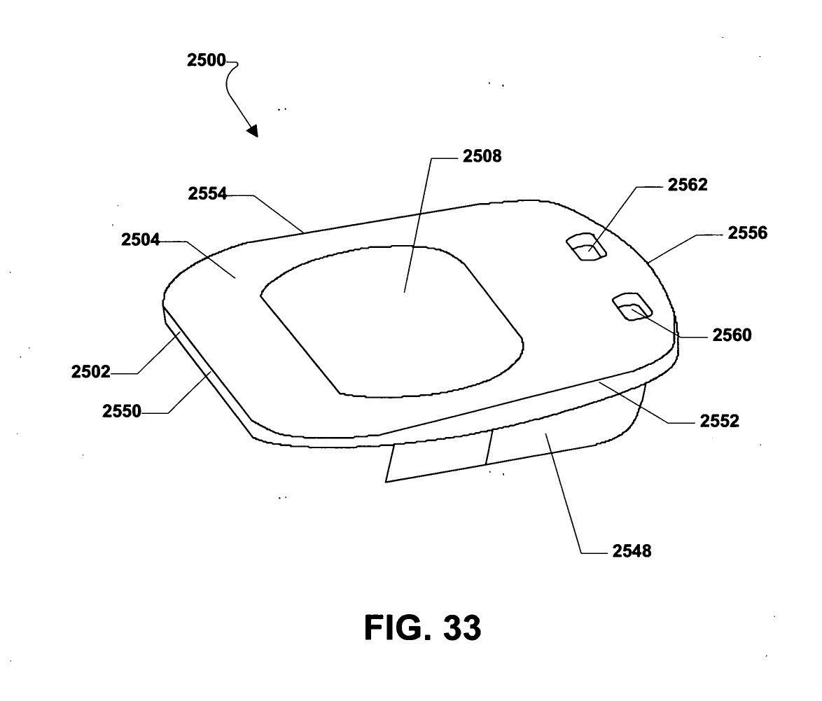

Referring to FIGS. 28 through 33 a third embodiment of an intervertebral prosthetic disc is shown and is generally designated 2300. As illustrated, the intervertebral prosthetic disc 2300 can include a superior component 2400, an inferior component 2500, and a nucleus 2600 disposed, or otherwise installed, therebetween. In a particular embodiment, the components 2400, 2500 and the nucleus 2600 can be made from one or more biocompatible materials. For example, the biocompatible materials can be one or more polymer materials.

In a particular embodiment, the superior component 2400 can include a superior support plate 2402 that has a superior articular surface 2404 and a superior bearing surface 2406. In a particular embodiment, the superior articular surface 2404 can be substantially flat and the superior bearing surface 2406 can be generally curved. In an alternative embodiment, at least a portion of the superior articular surface 2404 can be generally curved and the superior bearing surface 2406 can be substantially flat.

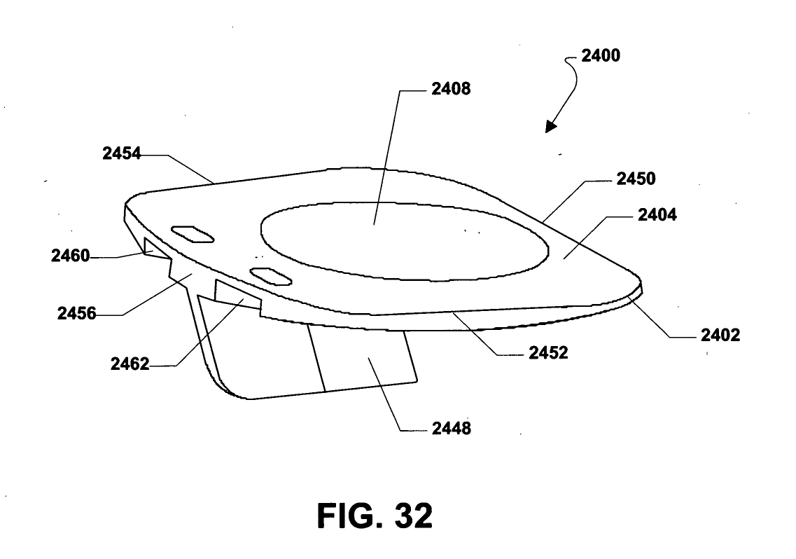

As illustrated in FIG. 32, a superior depression 2408 is established within the superior articular surface 2404 of the superior support plate 2402. In a particular embodiment, the superior depression 2408 has an arcuate shape. For example, the superior depression 2408 can have a hemispherical shape, an elliptical shape, a cylindrical shape, or any combination thereof.

FIG. 30 illustrates a cross-section of the nucleus 2600 configured to movably connect with the superior depression 2408. In a particular example, the nucleus 2600 is formed of a bulk polymeric material having a portion 2602 that is crosslinked to a greater extent than other portions of the bulk material. As illustrated, the portion 2602 is located in a posterior position relative to the intended placement of the prosthetic device 2300 in a patient. Alternatively, the portion 2602 can be located more centrally within the nucleus 2600, in an anterior location, to a left side, or to a right side of the nucleus 2600. Further, the extent to which the portion 2602 is crosslinked can be adapted to provide a desired mechanical property. Such a desired mechanical property can be determined based at least in part on a patient characteristic.

FIG. 28 through FIG. 32 indicate that the superior component 2400 can include a superior keel 2448 that extends from superior bearing surface 2406 and indicate that the inferior component 2500 can include an inferior keel 2548 that extends form an inferior bearing surface 2506. During installation, described below, the superior keel 2448 or the inferior keel 2548 can at least partially engage a keel groove that can be established within a cortical rim of a superior vertebra. Further, the superior keel 2448 or the inferior keel 2548 can be coated with a bone-growth promoting substance, e.g., a hydroxyapatite coating formed of calcium phosphate. In a particular embodiment, the superior keel 2448 or the inferior keel 2548 do not include proteins, e.g., bone morphogenetic protein (BMP). Additionally, the superior keel 2448 or the inferior keel 2548 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 2400, depicted in FIG. 32, can be generally rectangular in shape. For example, the superior component 2400 can have a substantially straight posterior side 2450. A first substantially straight lateral side 2452 and a second substantially straight lateral side 2454 can extend substantially perpendicularly from the posterior side 2450 to an anterior side 2456. In a particular embodiment, the anterior side 2456 can curve outward such that the superior component 2400 is wider through the middle than along the lateral sides 2452, 2454. Further, in a particular embodiment, the lateral sides 2452, 2454 are substantially the same length.

FIG. 31 shows that the superior component 2400 can include a first implant inserter engagement hole 2460 and a second implant inserter engagement hole 2462. In a particular embodiment, the implant inserter engagement holes 2460, 2462 are configured to receive a correspondingly shaped arm that extends from an implant inserter (not shown) that can be used to facilitate the proper installation of an intervertebral prosthetic disc, e.g., the intervertebral prosthetic disc 2300 shown in FIG. 28 through FIG. 32.

In a particular embodiment, the inferior component 2500 can include an inferior support plate 2502 that has an inferior articular surface 2504 and an inferior bearing surface 2506. In a particular embodiment, the inferior articular surface 2504 can be substantially flat and the inferior bearing surface 2506 can be generally curved. In an alternative embodiment, at least a portion of the inferior articular surface 2504 can be generally curved and the inferior bearing surface 2506 can be substantially flat.

In a particular embodiment, after installation, the superior bearing surface 2406 or the inferior bearing surface 2506 can be in direct contact with vertebral bone, e.g., cortical bone and cancellous bone. Further, the superior bearing surface 2406 or the inferior bearing surface 2506 can be coated with a bone-growth promoting substance, e.g., a hydroxyapatite coating formed of calcium phosphate. Additionally, the superior bearing surface 2406 or the inferior bearing surface 2506 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 30 and FIG. 32, an inferior depression 2508 is established within the inferior articular surface 2504 of the inferior support plate 2502. In a particular embodiment, the inferior depression 2508 has an arcuate shape. For example, the inferior depression 2508 can have a hemispherical shape, an elliptical shape, a cylindrical shape, or any combination thereof.

In a particular embodiment, the inferior component 2500, shown in FIG. 32, can be shaped to match the shape of the superior component 2400, shown in FIG. 32. Further, the inferior component 2500 can be generally rectangular in shape. For example, the inferior component 2500 can have a substantially straight posterior side 2550. A first substantially straight lateral side 2552 and a second substantially straight lateral side 2554 can extend substantially perpendicularly from the posterior side 2550 to an anterior side 2556. In a particular embodiment, the anterior side 2556 can curve outward such that the inferior component 2500 is wider through the middle than along the lateral sides 2552, 2554. Further, in a particular embodiment, the lateral sides 2552, 2554 are substantially the same length.

FIG. 31 shows that the inferior component 2500 can include a first implant inserter engagement hole 2560 and a second implant inserter engagement hole 2562. In a particular embodiment, the implant inserter engagement holes 2560, 2562 are configured to receive a correspondingly shaped arm that extends from an implant inserter (not shown) that can be used to facilitate the proper installation of an intervertebral prosthetic disc, e.g., the intervertebral prosthetic disc 2300 shown in FIG. 28 through FIG. 32.

In a particular embodiment, the overall height of the intervertebral prosthetic device 2300 can be in a range from fourteen millimeters to forty-six millimeters (14-46 mm). Further, the installed height of the intervertebral prosthetic device 2300 can be in a range from eight millimeters to sixteen millimeters (8-16 mm). In a particular embodiment, the installed height can be substantially equivalent to the distance between an inferior vertebra and a superior vertebra when the intervertebral prosthetic device 2300 is installed therebetween.

In a particular embodiment, the length of the intervertebral prosthetic device 2300, e.g., along a longitudinal axis, can be in a range from thirty millimeters to forty millimeters (30-40 mm). Additionally, the width of the intervertebral prosthetic device 2300, e.g., along a lateral axis, can be in a range from twenty-five millimeters to forty millimeters (25-40 mm).

Description of a Fourth Embodiment of an Intervertebral Prosthetic Disc

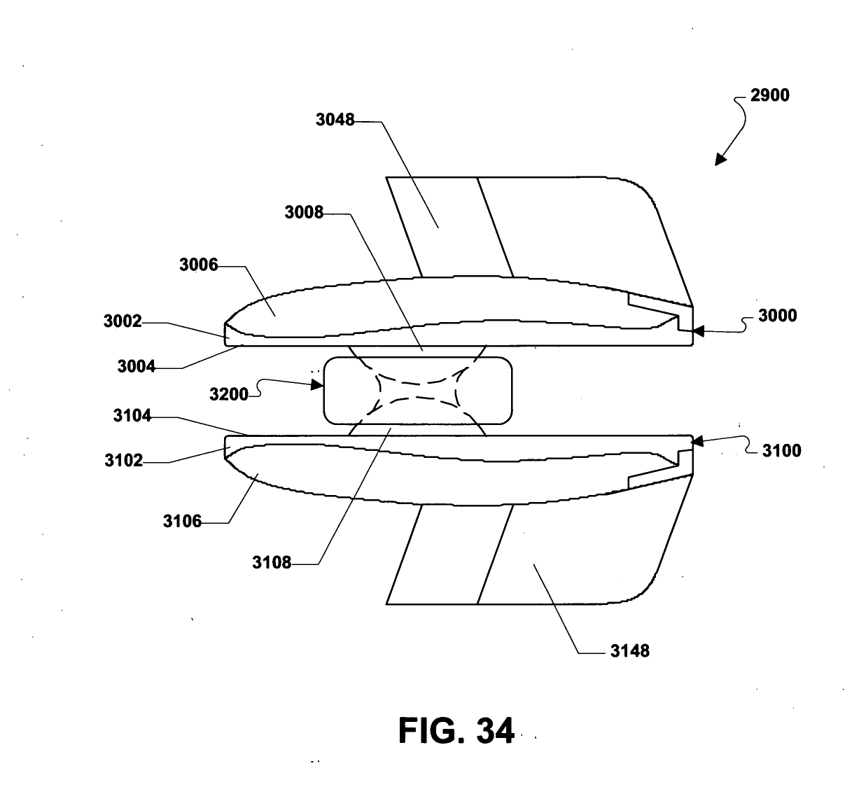

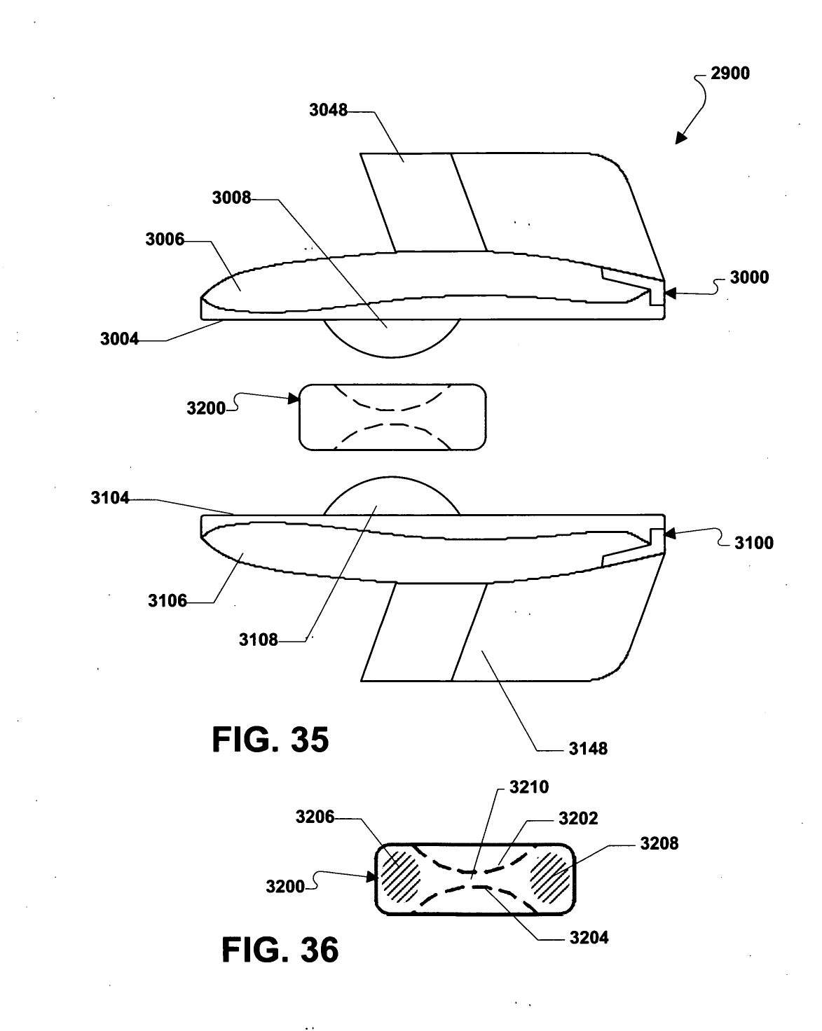

Referring to FIGS. 34 through 39, a fourth embodiment of an intervertebral prosthetic disc is shown and is generally designated 2900. As illustrated, the intervertebral prosthetic disc 2900 can include a superior component 3000, an inferior component 3100, and a nucleus 3200 disposed, or otherwise installed, therebetween. In a particular embodiment, the components 3000, 3100 and the nucleus 3200 can be made from one or more biocompatible materials. For example, the biocompatible materials can be one or more polymer materials.

In a particular embodiment, the superior component 3000 can include a superior support plate 3002 that has a superior articular surface 3004 and a superior bearing surface 3006. In a particular embodiment, the superior articular surface 3004 can be substantially flat and the superior bearing surface 3006 can be generally curved. In an alternative embodiment, at least a portion of the superior articular surface 3004 can be generally curved and the superior bearing surface 3006 can be substantially flat.

As illustrated in FIG. 34 through FIG. 38, a superior projection 3008 extends from the superior articular surface 3004 of the superior support plate 3002. In a particular embodiment, the superior projection 3008 has an arcuate shape. For example, the superior depression 3008 can have a hemispherical shape, an elliptical shape, a cylindrical shape, or any combination thereof.

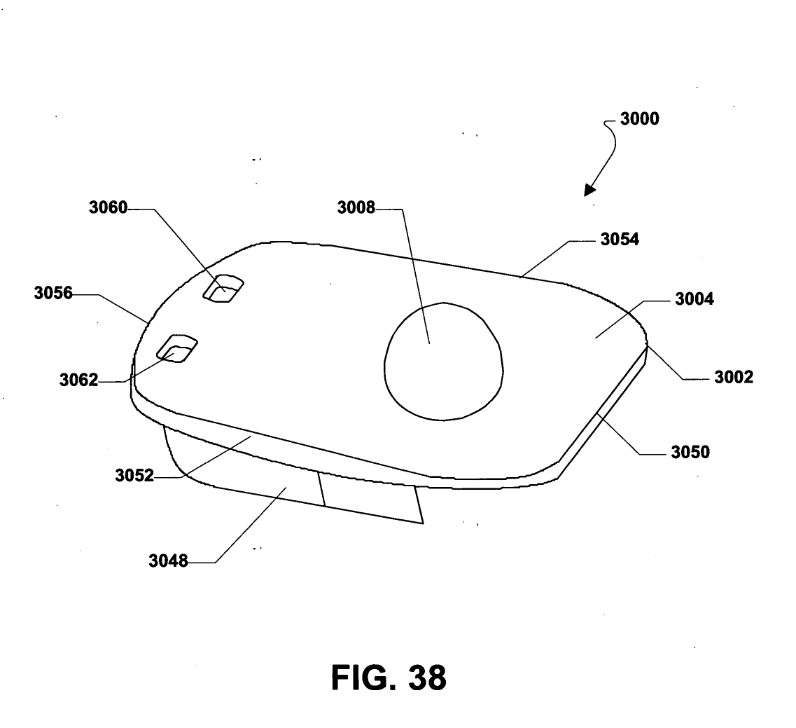

In a particular embodiment, the superior component 3000, depicted in FIG. 38, can be generally rectangular in shape. For example, the superior component 3000 can have a substantially straight posterior side 3050. A first substantially straight lateral side 3052 and a second substantially straight lateral side 3054 can extend substantially perpendicularly from the posterior side 3050 to an anterior side 3056. In a particular embodiment, the anterior side 3056 can curve outward such that the superior component 3000 is wider through the middle than along the lateral sides 3052, 3054. Further, in a particular embodiment, the lateral sides 3052, 3054 are substantially the same length.

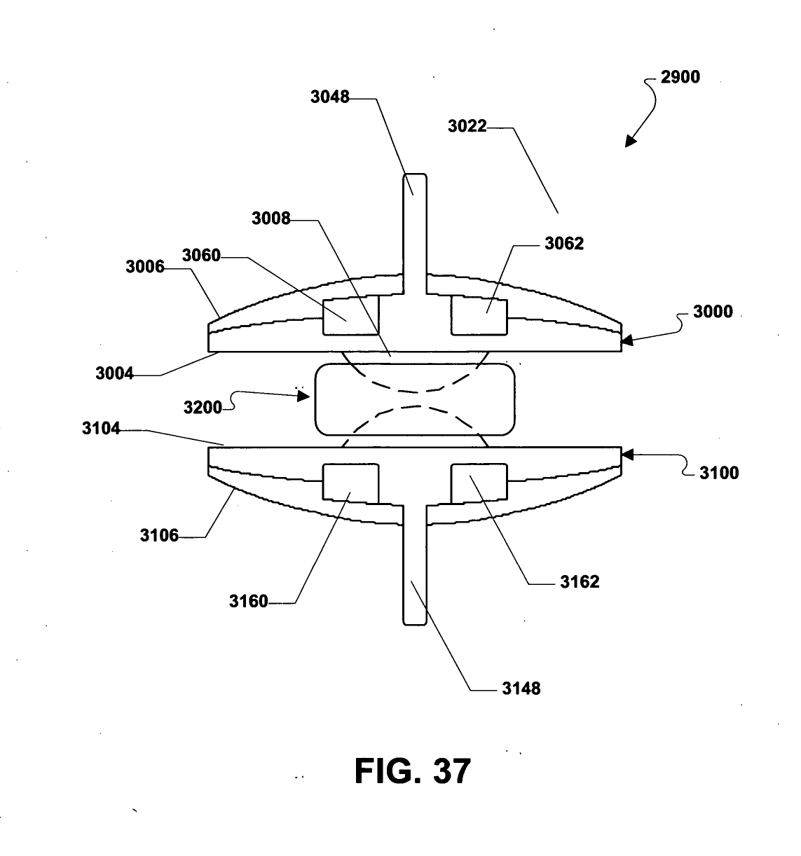

FIG. 37 shows that the superior component 3000 can include a first implant inserter engagement hole 3060 and a second implant inserter engagement hole 3062. In a particular embodiment, the implant inserter engagement holes 3060, 3062 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 34 through FIG. 39.

In a particular embodiment, the inferior component 3100 can include an inferior support plate 3102 that has an inferior articular surface 3104 and an inferior bearing surface 3106. In a particular embodiment, the inferior articular surface 3104 can be substantially flat and the inferior bearing surface 3106 can be generally curved. In an alternative embodiment, at least a portion of the inferior articular surface 3104 can be generally curved and the inferior bearing surface 3106 can be substantially flat.

In a particular embodiment, after installation, the superior bearing surface 3006 or the inferior bearing surface 3106 can be in direct contact with vertebral bone, e.g., cortical bone and cancellous bone. Further, the superior bearing surface 3006 or the inferior bearing surface 3106 can be coated with a bone-growth promoting substance, e.g., a hydroxyapatite coating formed of calcium phosphate. Additionally, the superior bearing surface 3006 or the inferior bearing surface 3106 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 34 through FIG. 37 and FIG. 39, an inferior projection 3108 can extend from the inferior articular surface 3104 of the inferior support plate 3102. In a particular embodiment, the inferior projection 3108 has an arcuate shape. For example, the inferior projection 3108 can have a hemispherical shape, an elliptical shape, a cylindrical shape, or any combination thereof.

FIG. 34 through FIG. 37 and FIG. 39 indicate that the superior component 3000 can include a superior keel 3048 that extends from superior bearing surface 3006 and indicate that the inferior component 3100 can include an inferior keel 3148 that extends from inferior bearing surface 3106. During installation, described below, the superior keel 3048 or the inferior keel 3148 can at least partially engage a keel groove that can be established within a cortical rim of a vertebra. Further, the superior keel 3048 or the inferior keel 3148 can be coated with a bone-growth promoting substance, e.g., a hydroxyapatite coating formed of calcium phosphate. In a particular embodiment, the superior keel 3048 or the inferior keel 3148 do not include proteins, e.g., bone morphogenetic protein (BMP). Additionally, the superior keel 3048 or the inferior keel 3148 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

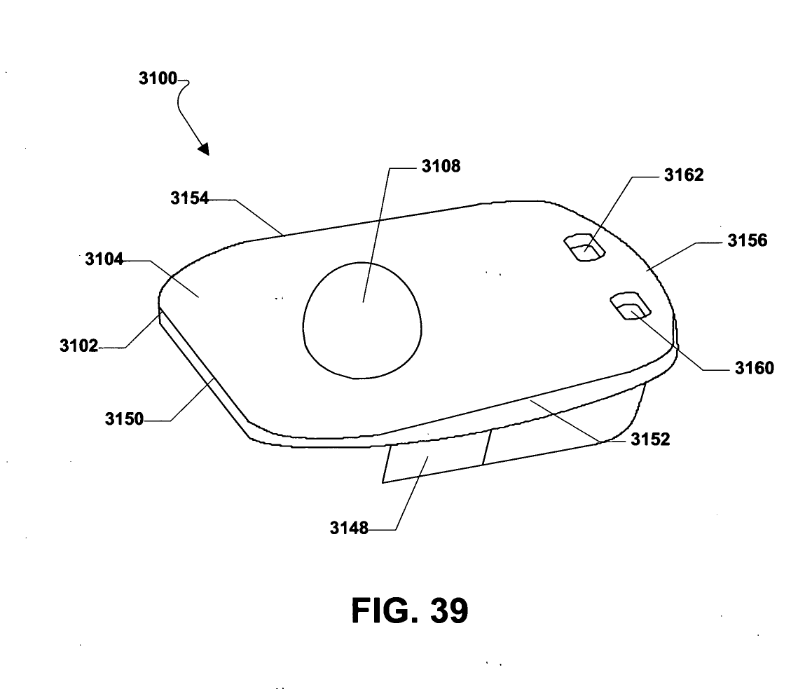

In a particular embodiment, the inferior component 3100, shown in FIG. 39, can be shaped to match the shape of the superior component 3000, shown in FIG. 38. Further, the inferior component 3100 can be generally rectangular in shape. For example, the inferior component 3100 can have a substantially straight posterior side 3150. A first substantially straight lateral side 3152 and a second substantially straight lateral side 3154 can extend substantially perpendicularly from the posterior side 3150 to an anterior side 3156. In a particular embodiment, the anterior side 3156 can curve outward such that the inferior component 3100 is wider through the middle than along the lateral sides 3152, 3154. Further, in a particular embodiment, the lateral sides 3152, 3154 are substantially the same length.

FIG. 37 shows that the inferior component 3100 can include a first implant inserter engagement hole 3160 and a second implant inserter engagement hole 3162. In a particular embodiment, the implant inserter engagement holes 3160, 3162 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 34 through FIG. 39.

FIG. 36 shows that the nucleus 3200 can include a superior depression 3202 and an inferior depression 3204. In a particular embodiment, the superior depression 3202 and the inferior depression 3204 can each have an arcuate shape. For example, the superior depression 3202 of the nucleus 3200 and the inferior depression 3204 of the nucleus 3200 can have a hemispherical shape, an elliptical shape, a cylindrical shape, or any combination thereof. Further, in a particular embodiment, the superior depression 3202 can be curved to match the superior projection 3008 of the superior component 3000. Also, in a particular embodiment, the inferior depression 3204 of the nucleus 3200 can be curved to match the inferior projection 3108 of the inferior component 3100.

FIG. 36 illustrates that the nucleus 3200 can include a portion 3206 or a portion 3208 that are crosslinked to a greater extent than other portions of the nucleus 3200. As illustrated, the portions 3206 and 3208 represent posterior and anterior portions of the nucleus 3200, respectively. Alternatively, a center portion 3210 can be crosslinked to a greater extent than other portions, such as the portions 3206 and 3208. In this manner, portions can be crosslinked to impart desired mechanical properties to the nucleus 3200. While not illustrated, the superior and inferior projection 3008 and 3108 can be formed of crosslinkable bulk material. As such, these projections 3008 and 3108 can be crosslinked to an extent or at a portion that provides desired mechanical performance of the device 2900.

In a particular embodiment, the overall height of the intervertebral prosthetic device 2900 can be in a range from fourteen millimeters to forty-six millimeters (14-46 mm). Further, the installed height of the intervertebral prosthetic device 2900 can be in a range from eight millimeters to sixteen millimeters (8-16 mm). In a particular embodiment, the installed height can be substantially equivalent to the distance between an inferior vertebra and a superior vertebra when the intervertebral prosthetic device 2900 is installed therebetween.

In a particular embodiment, the length of the intervertebral prosthetic device 2900, e.g., along a longitudinal axis, can be in a range from thirty millimeters to forty millimeters (30-40 mm). Additionally, the width of the intervertebral prosthetic device 2900, e.g., along a lateral axis, can be in a range from twenty-five millimeters to forty millimeters (25-40 mm).

Description of a Fifth Embodiment of an Intervertebral Prosthetic Disc

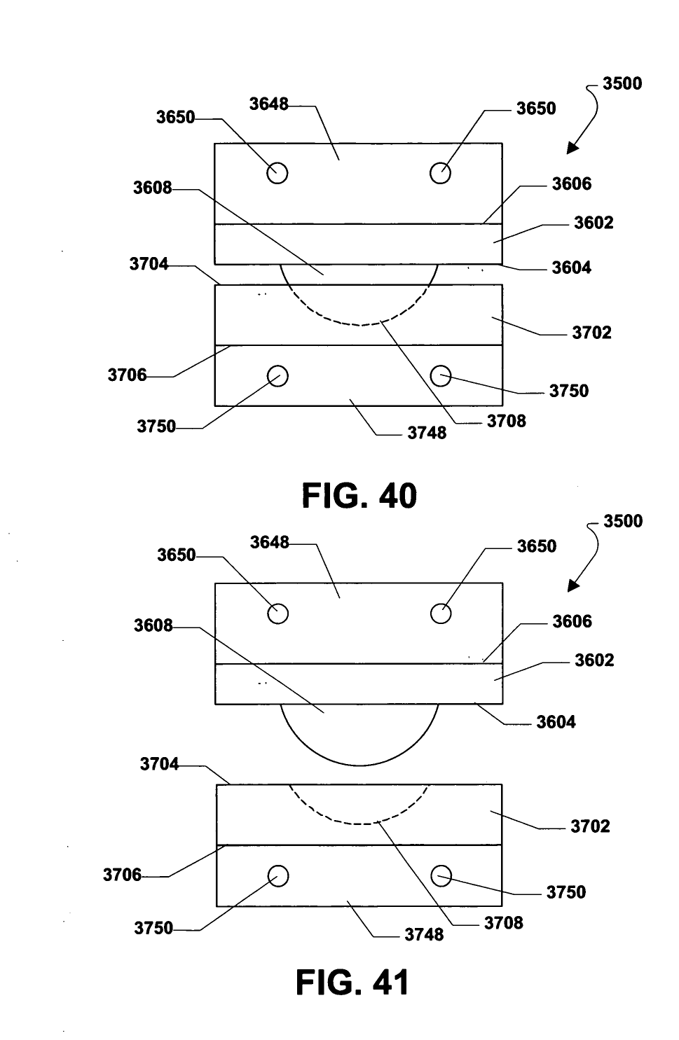

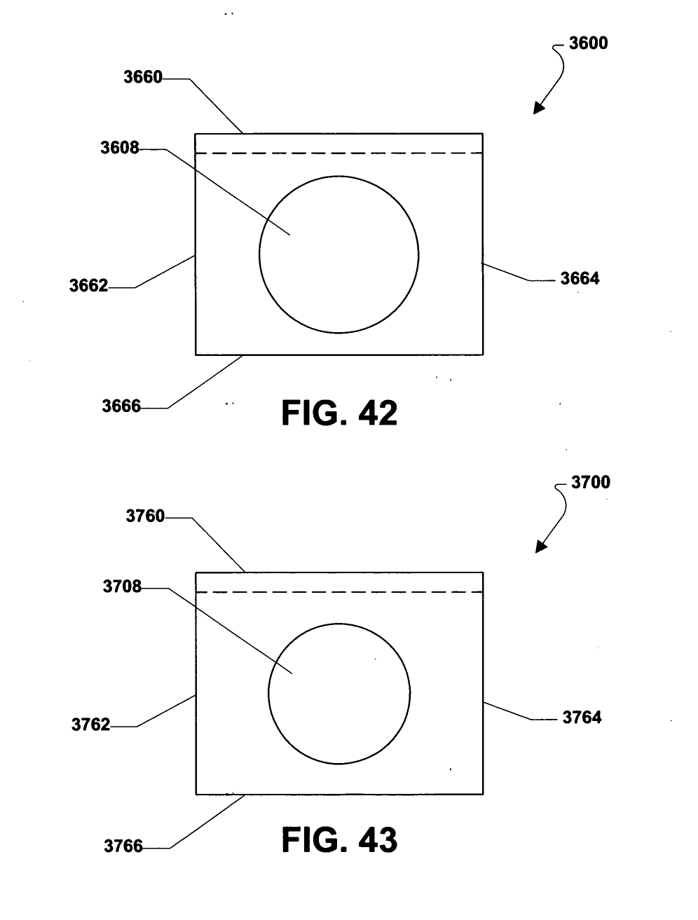

Referring to FIGS. 40 through 43 a fifth embodiment of an intervertebral prosthetic disc is shown and is generally designated 3500. As illustrated, the intervertebral prosthetic disc 3500 can include a superior component 3600 and an inferior component 3700. In a particular embodiment, the components 3600, 3700 can be made from one or more biocompatible materials. For example, the biocompatible materials can be one or more polymer materials.

In a particular embodiment, the superior component 3600 can include a superior support plate 3602 that has a superior articular surface 3604 and a superior bearing surface 3606. In a particular embodiment, the superior articular surface 3604 can be substantially flat and the superior bearing surface 3606 can be substantially flat. In an alternative embodiment, at least a portion of the superior articular surface 3604 can be generally curved and at least a portion of the superior bearing surface 3606 can be generally curved.

As illustrated in FIG. 40 through FIG. 42, a projection 3608 extends from the superior articular surface 3604 of the superior support plate 3602. In a particular embodiment, the projection 3608 has a hemispherical shape. Alternatively, the projection 3608 can have an elliptical shape, a cylindrical shape, or other arcuate shape.

FIG. 40 through FIG. 42 also show that the superior component 3600 can include a superior bracket 3648 that can extend substantially perpendicular from the superior support plate 3602. Further, the superior bracket 3648 can include at least one hole 3650. In a particular embodiment, a fastener, e.g., a screw, can be inserted through the hole 3650 in the superior bracket 3648 in order to attach, or otherwise affix, the superior component 3600 to a superior vertebra.

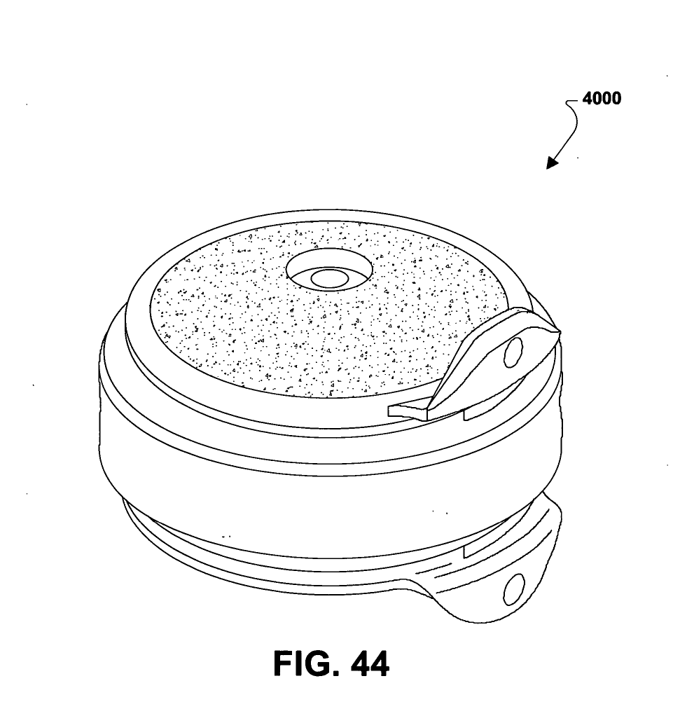

As illustrated in FIG. 43, the superior component 3600 can be generally rectangular in shape. For example, the superior component 3600 can have a substantially straight posterior side 3660. A first straight lateral side 3662 and a second substantially straight lateral side 3664 can extend substantially perpendicular from the posterior side 3660 to a substantially straight anterior side 3666. In a particular embodiment, the anterior side 3666 and the posterior side 3660 are substantially the same length. Further, in a particular embodiment, the lateral sides 3662, 3664 are substantially the same length.

In a particular embodiment, the inferior component 3700 can include an inferior support plate 3702 that has an inferior articular surface 3704 and an inferior bearing surface 3706. In a particular embodiment, the inferior articular surface 3704 can be generally curved and the inferior bearing surface 3706 can be substantially flat. In an alternative embodiment, the inferior articular surface 3704 can be substantially flat and at least a portion of the inferior bearing surface 3706 can be generally curved.

As illustrated in FIG. 40 through FIG. 42, a depression 3708 extends into the inferior articular surface 3704 of the inferior support plate 3702. In a particular embodiment, the depression 3708 is sized and shaped to receive the projection 3608 of the superior component 3600. For example, the depression 3708 can have a hemi-spherical shape. Alternatively, the depression 3708 can have an elliptical shape, a cylindrical shape, or other arcuate shape.

FIG. 40 through FIG. 42 also show that the inferior component 3700 can include an inferior bracket 3748 that can extend substantially perpendicular from the inferior support plate 3702. Further, the inferior bracket 3748 can include a hole 3750. In a particular embodiment, a fastener, e.g., a screw, can be inserted through the hole 3750 in the inferior bracket 3748 in order to attach, or otherwise affix, the inferior component 3700 to an inferior vertebra.

The superior bearing surface 3606 or the inferior bearing surface 3706 can be coated with a bone-growth promoting substance, e.g., a hydroxyapatite coating formed of calcium phosphate. Additionally, the superior bearing surface 3606 or the inferior bearing surface 3706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 43, the inferior component 3700 can be generally rectangular in shape. For example, the inferior component 3700 can have a substantially straight posterior side 3760. A first straight lateral side 3762 and a second substantially straight lateral side 3764 can extend substantially perpendicular from the posterior side 3760 to a substantially straight anterior side 3766. In a particular embodiment, the anterior side 3766 and the posterior side 3760 are substantially the same length. Further, in a particular embodiment, the lateral sides 3762, 3764 are substantially the same length.

In a particular embodiment, the overall height of the intervertebral prosthetic device 3500 can be in a range from fourteen millimeters to forty-six millimeters (14-46 mm). Further, the installed height of the intervertebral prosthetic device 3500 can be in a range from eight millimeters to sixteen millimeters (8-16 mm). In a particular embodiment, the installed height can be substantially equivalent to the distance between an inferior vertebra and a superior vertebra when the intervertebral prosthetic device 3500 is installed therebetween.

In a particular embodiment, the length of the intervertebral prosthetic device 3500, e.g., along a longitudinal axis, can be in a range from thirty millimeters to forty millimeters (30-40 mm). Additionally, the width of the intervertebral prosthetic device 3500, e.g., along a lateral axis, can be in a range from twenty-five millimeters to forty millimeters (25-40 mm). Moreover, in a particular embodiment, each bracket 3648, 3748 can have a height in a range from three millimeters to fifteen millimeters (3-15 mm).

In a further embodiment, the projection 3608 can be formed of a crosslinkable bulk polymeric material. A portion of the bulk polymeric material can be crosslinked to a greater extent than other portions of the bulk polymeric material. The crosslinking of the portion of the bulk polymeric material can be effected to provide a desired mechanical property for the projection 3608.

Description of a Sixth Embodiment of an Intervertebral Prosthetic Disc

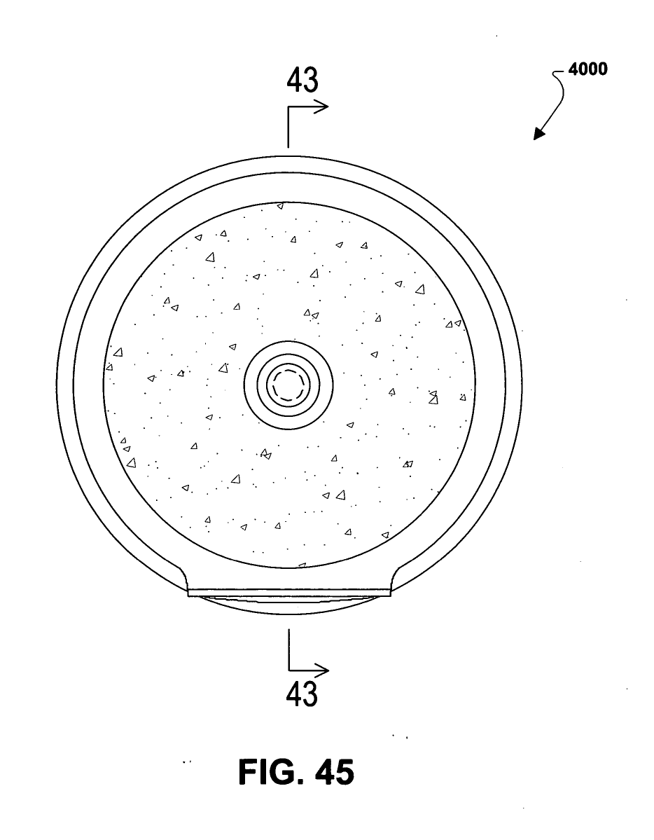

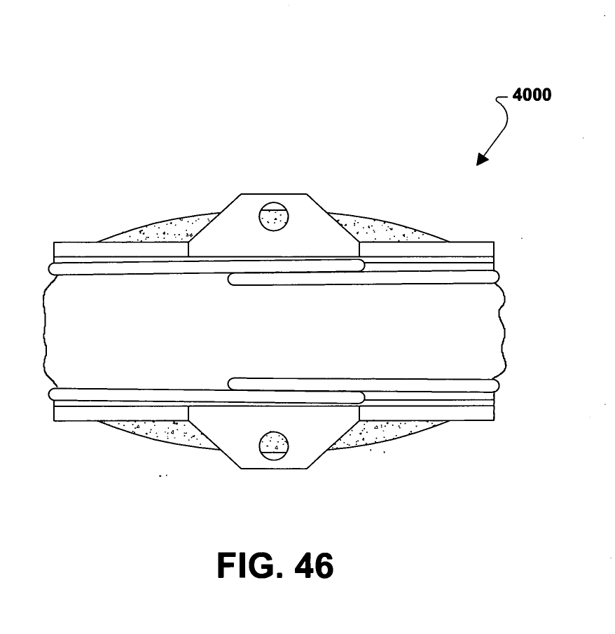

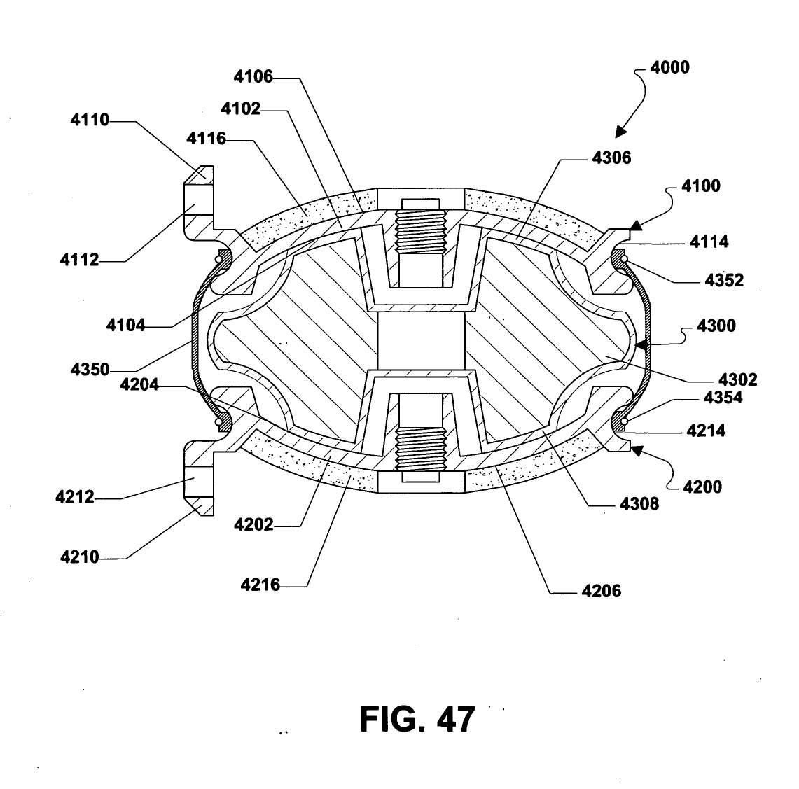

Referring to FIGS. 44 through 47, a sixth embodiment of an intervertebral prosthetic disc is shown and is generally designated 4000. As illustrated in FIG. 47, the intervertebral prosthetic disc 4000 can include a superior component 4100, an inferior component 4200, and a nucleus 4300 disposed, or otherwise installed, therebetween. In a particular embodiment, a sheath 4350 surrounds the nucleus 4300 and is affixed or otherwise coupled to the superior component 4100 and the inferior component 4200. In a particular embodiment, the components 4100, 4200 and the nucleus 4300 can be made from one or more biocompatible materials. For example, the biocompatible materials can be one or more polymer materials.

In a particular embodiment, the superior component 4100 can include a superior support plate 4102 that has a superior articular surface 4104 and a superior bearing surface 4106. In a particular embodiment, the superior support plate 4102 can be generally rounded, generally cup shaped, or generally bowl shaped. Further, in a particular embodiment, the superior articular surface 4104 can be generally rounded or generally curved and the superior bearing surface 4106 can be generally rounded or generally curved.

FIG. 47 also shows that the superior support plate 4102 can include a superior bracket 4110 that can extend substantially perpendicular from the superior support plate 4102. The superior bracket 4110 can include a hole 4112. In a particular embodiment, a fastener, e.g., a screw, can be inserted through the hole 4112 in the superior bracket 4110 in order to attach, or otherwise affix, the superior component 4100 to a superior vertebra.

Moreover, the superior support plate 4102 includes a superior channel 4114 established around the perimeter of the superior support plate 4102. In a particular embodiment, a portion of the sheath 4300 can be held within the superior channel 4114 using a superior retaining ring 4352.

In a particular embodiment, the inferior component 4200 can include an inferior support plate 4202 that has an inferior articular surface 4204 and an inferior bearing surface 4206. In a particular embodiment, the inferior support plate 4202 can be generally rounded, generally cup shaped, or generally bowl shaped. Further, in a particular embodiment, the inferior articular surface 4204 can be generally rounded or generally curved and the inferior bearing surface 4206 can be generally rounded or generally curved.

FIG. 47 also shows that the inferior support plate 4202 can include an inferior bracket 4210 that can extend substantially perpendicular from the inferior support plate 4202. The inferior bracket 4210 can include a hole 4212. In a particular embodiment, a fastener, e.g., a screw, can be inserted through the hole 4212 in the inferior bracket 4210 in order to attach, or otherwise affix, the inferior component 4200 to an inferior vertebra.

Moreover, the inferior support plate 4202 includes an inferior channel 4214 established around the perimeter of the inferior support plate 4202. In a particular embodiment, a portion of the sheath 4300 can be held within the inferior channel 4214 using an inferior retaining ring 4354.

As depicted in FIG. 47, the superior support plate 4102 can include a bone growth promoting layer 4116 disposed, or otherwise deposited, on the superior bearing surface 4106 and the inferior support plate 4202 can include a bone growth promoting layer 4216 disposed, or otherwise deposited, on the inferior bearing surface 4206. In a particular embodiment, the bone growth promoting layers 4416 and 4216 can include a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As depicted in FIG. 47, the nucleus 4300 can be generally toroid shaped. Further, the nucleus 4300 includes a core 4302 and an outer wear resistant layer 4304. In a particular embodiment, the core 4302 of the nucleus can be made from one or more biocompatible materials. For example, the biocompatible materials can be one or more polymer materials, described herein. Further, the outer wear resistant layer 4304 can be established by crosslinking the surface of the core 4302.

In addition, the core 4302 can be formed of a bulk material that can include a portion that is crosslinked to a greater extent than other portions. For example, a portion of the toroid shaped nucleus 4300 that is posterior can be crosslinked to a greater extent than portions that are more anterior. Alternatively, anterior portions can be crosslinked. In a further example, portions that are between the anterior and posterior positions can be crosslinked to a greater extent than anterior or posterior portions.

Description of a Nucleus Implant

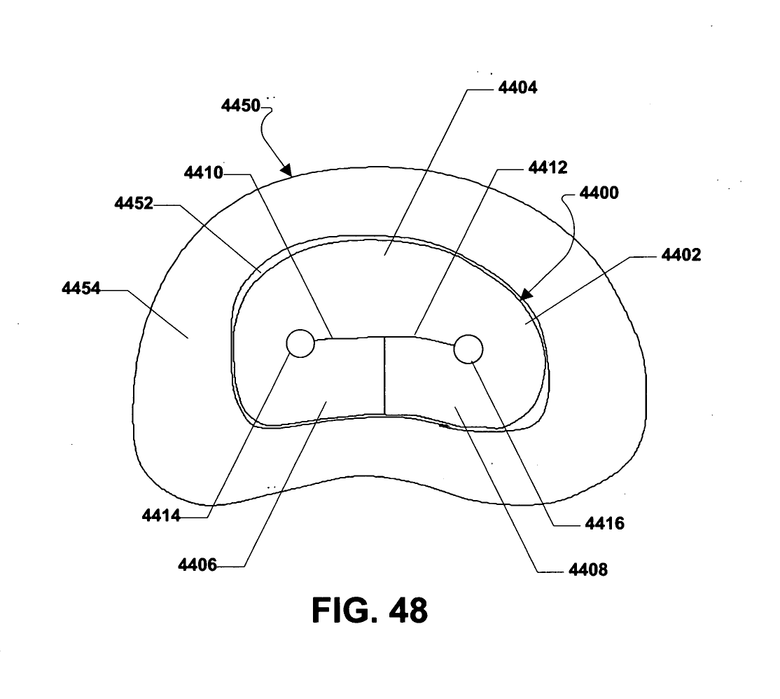

Referring to FIG. 48 through FIG. 51, an embodiment of a nucleus implant is shown and is designated 4400. As shown, the nucleus implant 4400 can include a load bearing elastic body 4402. The load bearing elastic body 4402 can include a central portion 4404. A first end 4406 and a second end 4408 can extend from the central portion 4404 of the load bearing elastic body 4402.

As depicted in FIG. 48, the first end 4406 of the load bearing elastic body 4402 can establish a first fold 4410 with respect to the central portion 4404 of the load bearing elastic body 4402. Further, the second end 4408 of the load bearing elastic body 4402 can establish a second fold 4412 with respect to the central portion 4404 of the load bearing elastic body 4402. In a particular embodiment, the ends 4406, 4408 of the load bearing elastic body 4402 can be folded toward each other relative to the central portion 4404 of the load bearing elastic body 4402. Also, when folded, the ends 4406, 4408 of the load bearing elastic body 4402 are parallel to the central portion 4404 of the load bearing elastic body 4402. Further, in a particular embodiment, the first fold 4410 can define a first aperture 4414 and the second fold 4412 can define a second aperture 4416. In a particular embodiment, the apertures 4414, 4416 are generally circular. However, the apertures 4414, 4416 can have any arcuate shape.

In an exemplary embodiment, the nucleus implant 4400 can have a rectangular cross-section with sharp or rounded corners. Alternatively, the nucleus implant 4400 can have a circular cross-section. As such, the nucleus implant 4400 may form a rectangular prism or a cylinder.

FIG. 48 indicates that the nucleus implant 4400 can be implanted within an intervertebral disc 4450 between a superior vertebra and an inferior vertebra. More specifically, the nucleus implant 4400 can be implanted within an intervertebral disc space 4452 established within the annulus fibrosis 4454 of the intervertebral disc 4450. The intervertebral disc space 4452 can be established by removing the nucleus pulposus (not shown) from within the annulus fibrosis 4454.

In a particular embodiment, the nucleus implant 4400 can provide shock-absorbing characteristics substantially similar to the shock absorbing characteristics provided by a natural nucleus pulposus. Additionally, in a particular embodiment, the nucleus implant 4400 can have a height that is sufficient to provide proper support and spacing between a superior vertebra and an inferior vertebra.

In a particular embodiment, the nucleus implant 4400 shown in FIG. 48 can have a shape memory and the nucleus implant 4400 can be configured to allow extensive short-term manual, or other, deformation without permanent deformation, cracks, tears, breakage or other damage, that can occur, for example, during placement of the implant into the intervertebral disc space 4452.

For example, the nucleus implant 4400 can be deformable, or otherwise configurable, e.g., manually, from a folded configuration, shown in FIG. 48, to a substantially straight configuration, shown in FIG. 48, in which the ends 4406, 4408 of the load bearing elastic body 4402 are substantially aligned with the central portion 4404 of the load bearing elastic body 4402. In a particular embodiment, when the nucleus implant 4400 the folded configuration, shown in FIG. 48, can be considered a relaxed state for the nucleus implant 4400. Also, the nucleus implant 4400 can be placed in the straight configuration for placement, or delivery into an intervertebral disc space within an annulus fibrosis.

In a particular embodiment, the nucleus implant 4400 can include a shape memory, and as such, the nucleus implant 4400 can automatically return to the folded, or relaxed, configuration from the straight configuration after force is no longer exerted on the nucleus implant 4400. Accordingly, the nucleus implant 4400 can provide improved handling and manipulation characteristics since the nucleus implant 4400 can be deformed, configured, or otherwise handled, by an individual without resulting in any breakage or other damage to the nucleus implant 4400.

Although the nucleus implant 4400 can have a wide variety of shapes, the nucleus implant 4400 when in the folded, or relaxed, configuration can conform to the shape of a natural nucleus pulposus. As such, the nucleus implant 4400 can be substantially elliptical when in the folded, or relaxed, configuration. In one or more alternative embodiments, the nucleus implant 4400, when folded, can be generally annular-shaped or otherwise shaped as required to conform to the intervertebral disc space within the annulus fibrosis. Moreover, when the nucleus implant 4400 is in an unfolded, or non-relaxed, configuration, such as the substantially straightened configuration, the nucleus implant 4400 can have a wide variety of shapes. For example, the nucleus implant 4400, when straightened, can have a generally elongated shape. Further, the nucleus implant 4400 can have a cross section that is: generally elliptical, generally circular, generally rectangular, generally square, generally triangular, generally trapezoidal, generally rhombic, generally quadrilateral, any generally polygonal shape, or any combination thereof.

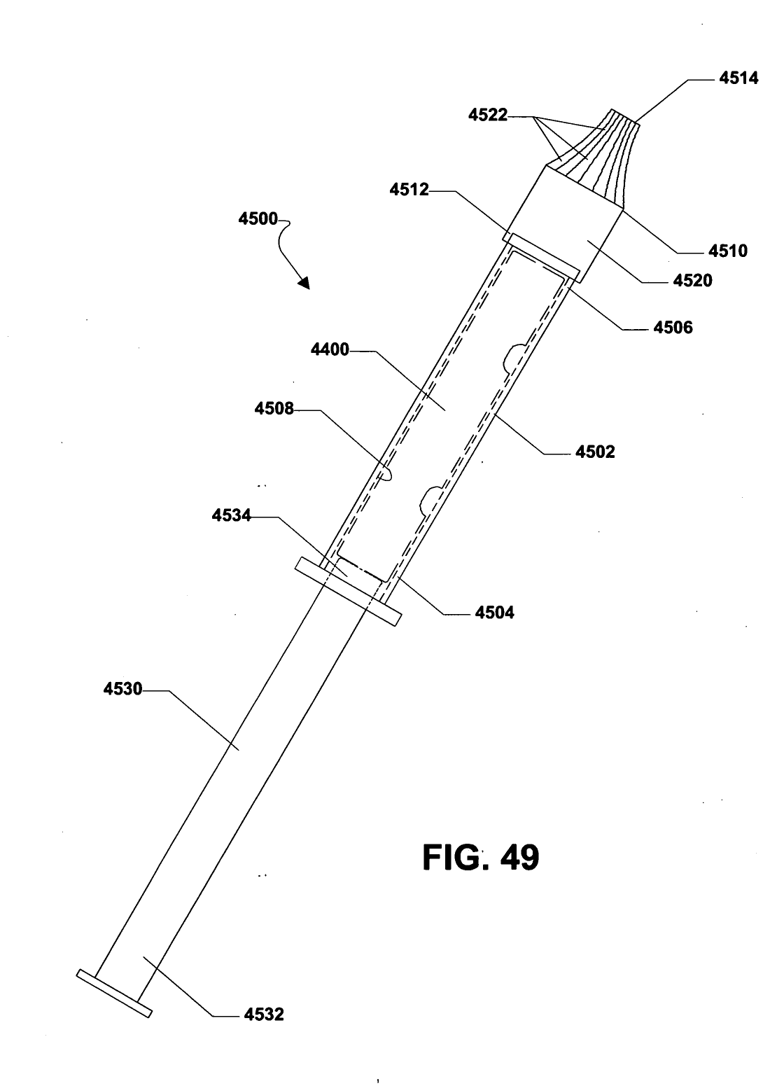

Referring to FIG. 49, a nucleus delivery device is shown and is generally designated 4500. As illustrated in FIG. 49, the nucleus delivery device 4500 can include an elongated housing 4502 that can include a proximal end 4504 and a distal end 4506. The elongated housing 4502 can be hollow and can form an internal cavity 4508. As depicted in FIG. 49, the nucleus delivery device 4500 can also include a tip 4510 having a proximal end 4512 and a distal end 4514. In a particular embodiment, the proximal end 4512 of the tip 4510 can be affixed, or otherwise attached, to the distal end 4506 of the housing 4502.

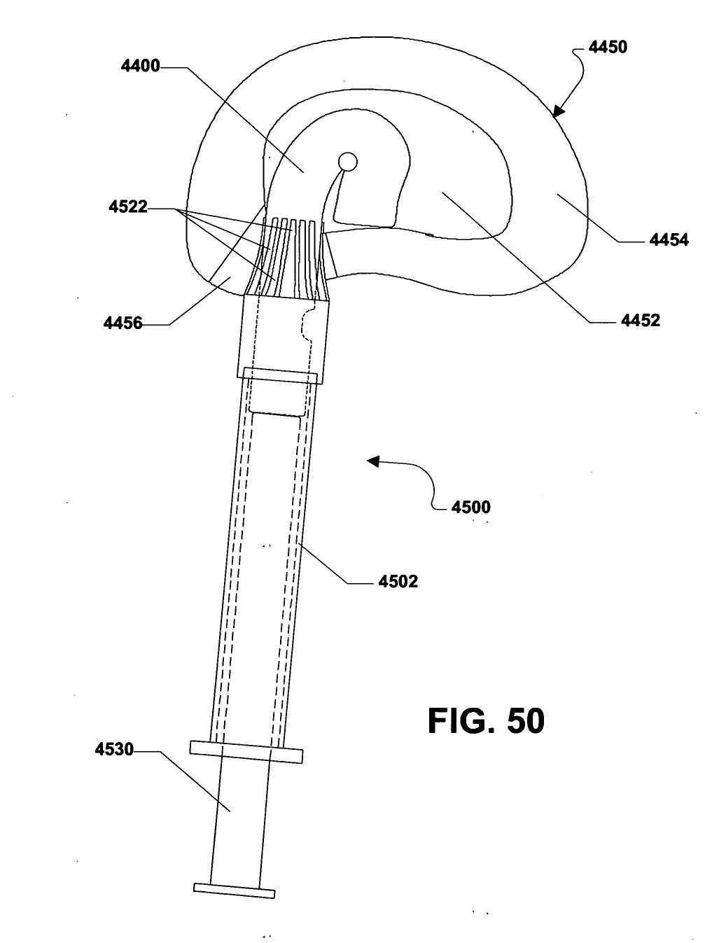

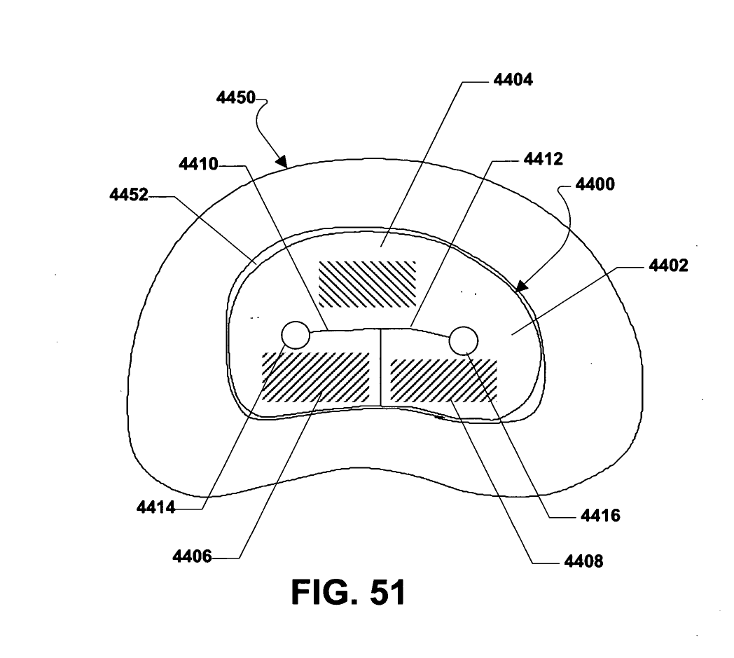

In a particular embodiment, the tip 4510 of the nucleus delivery device 4500 can include a generally hollow base 4520. Further, a plurality of movable members 4522 can be attached to the base 4520 of the tip 4510. The movable members 4522 are movable between a closed position, shown in FIG. 49, and an open position, shown in FIG. 50, as a nucleus implant is delivered using the nucleus delivery device 4500 as described below.

FIG. 49 further shows that the nucleus delivery device 4500 can include a generally elongated plunger 4530 that can include a proximal end 4532 and a distal end 4534. In a particular embodiment, the plunger 4530 can be sized and shaped to slidably fit within the housing 4502, e.g., within the cavity 4508 of the housing 4502.

As shown in FIG. 49 and FIG. 50, a nucleus implant, e.g., the nucleus implant 4400 shown in FIG. 49, can be disposed within the housing 4502, e.g., within the cavity 4508 of the housing 4502. Further, the plunger 4530 can slide within the cavity 4508, relative to the housing 4502, in order to force the nucleus implant 4400 from within the housing 4502 and into the intervertebral disc space 4452. As shown in FIG. 50, as the nucleus implant 4400 exits the nucleus delivery device 4500, the nucleus implant 4400 can move from the non-relaxed, straight configuration to the relaxed, folded configuration within the annulus fibrosis. Further, as the nucleus implant 4400 exits the nucleus delivery device 4500, the nucleus implant 4400 can cause the movable members 4522 to move to the open position, as shown in FIG. 50.

In a particular embodiment, the nucleus implant 4400 can be installed using a posterior surgical approach, as shown. Further, the nucleus implant 4400 can be installed through a posterior incision 4456 made within the annulus fibrosis 4454 of the intervertebral disc 4450. Alternatively, the nucleus implant 4400 can be installed using an anterior surgical approach, a lateral surgical approach, or any other surgical approach well known in the art.

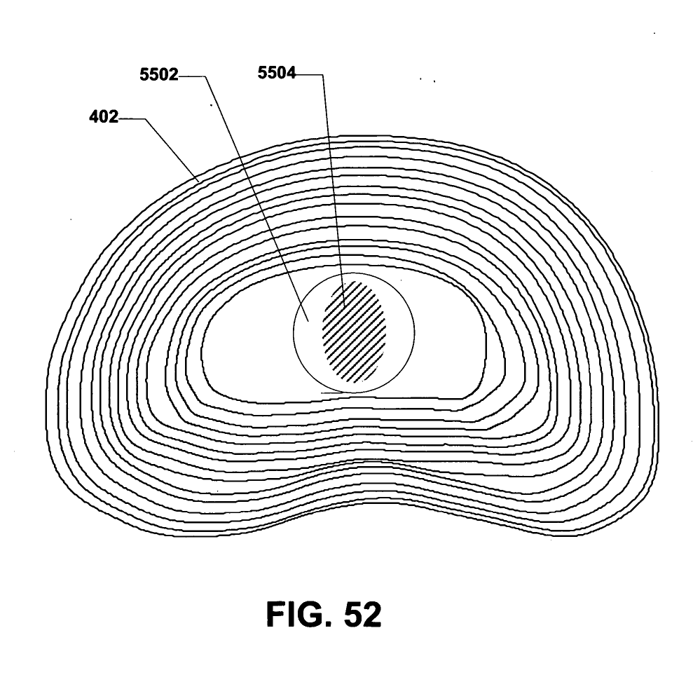

Referring to FIG. 51, the load bearing elastic body 4402 is illustrated as including a first end 4406, a second end 4408, and a central region 4404. In a particular embodiment, the bulk polymeric material at the first end 4406 and at the second end 4408 can be crosslinked to a greater extent than at the central portion 4404. Alternatively, the bulk polymeric material at the central portion 4404 can be crosslinked to a greater extent than the bulk polymeric material at the first end 4406 or the second end 4408. Such crosslinking can be effected during manufacture or within the delivery device 4500 prior to implanting.

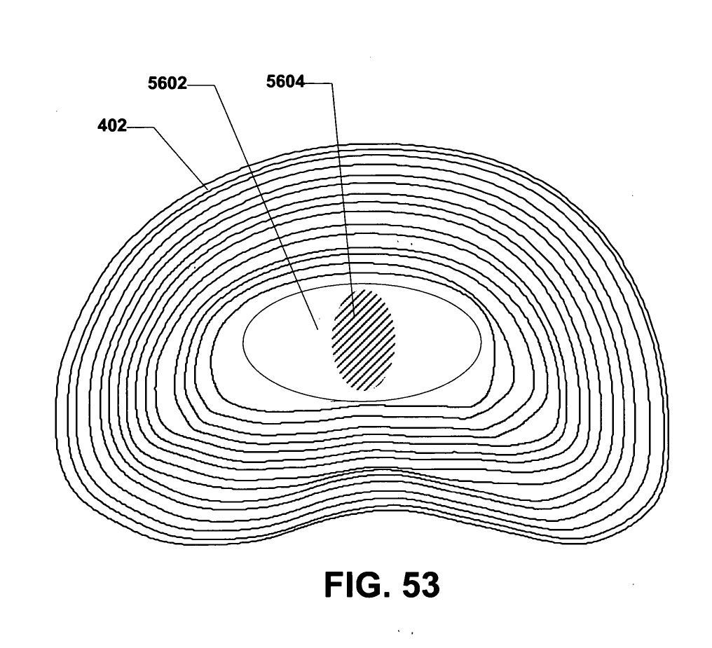

Referring to FIG. 52 and FIG. 53, a load bearing elastic body, such as a load bearing body 5502 illustrated in FIG. 52 or a load bearing body 5602 illustrated in FIG. 53, can be inserted between two vertebrae into a region formerly occupied by the nucleus pulposus 404 and surrounded by the annulus fibrosis 402. In the embodiment illustrated in FIG. 52, the load bearing body 5502 is spherical in shape. In an alternative embodiment illustrated in FIG. 53, the load bearing body 5602 can have an elliptical shape. Alternatively, the load bearing body can have a spheroidal shape, an ellipsoidal shape, a cylindrical shape, a polygonal prism shape, a tetrahedral shape, a frustoconical shape, or any combination thereof. In a particular embodiment, the load bearing body can include a stabilizer, such as a stabilizer in the shape of a disc extending radially from an axially central location of the load bearing body.

In an exemplary embodiment, the load bearing body, such as the load bearing body 5502 illustrated in FIG. 52 or the load bearing body 5602 illustrated in FIG. 53, can have a maximum radius that is greater than the distance between the two vertebrae between which the load bearing body is to be implanted. Alternatively, the maximum radius can be equal to or less than the distance between the two vertebrae between which the load bearing body is to be implanted. In a particular embodiment, the maximum radius of the load bearing body can be between about 3 mm to about 15 mm.

In a particular embodiment, the elastic body, such as the elastic body 5502 illustrated in FIG. 52 or the load bearing body 5602 illustrated in FIG. 53, is formed of a crosslinkable polymeric bulk material. A portion of the bulk polymeric material can be crosslinked to provide a desired mechanical performance. For example, the bulk polymeric material of the load bearing body 5502 can be crosslinked in a center portion 5504, as illustrated in FIG. 52. Alternatively, the bulk polymeric material of the load bearing body 5502 can be crosslinked at a left portion, a right portion, an anterior portion, a posterior portion, a top portion, a bottom portion, or any combination thereof. In another example, the bulk polymeric material of the load bearing body 5602 can be crosslinked in a center portion 5604, as illustrated in FIG. 53. Alternatively, the bulk polymeric material of the load bearing body 5602 can be crosslinked at a left portion, a right portion, an anterior portion, a posterior portion, a top portion, a bottom portion, or any combination thereof. In a further embodiment, a core of the load bearing body, such as the load bearing body 5502 of FIG. 52 or the load bearing body 5602 of FIG. 53, can be crosslinked and a surface not crosslinked or crosslinked to a lesser extent. Such an embodiment can provide a hard articulate shape, while limiting slipping of the component.

### CONCLUSION

With the configuration of structure described above, the intervertebral prosthetic disc or nucleus implant according to one or more of the embodiments provides a device that can be implanted to replace at least a portion of a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion therebetween.

In a particular embodiment, the crosslinked portions of a bulk polymer material used in forming one or more of the component of the exemplary intervertebral prosthetic discs described herein can provide improved mechanical performance. Accordingly, comfort to a patient, range of motion, and performance of the prosthetic disc can be improved. In addition, crosslinking of a portion of the bulk polymeric material of a component can reduce creep and flow caused by stress, while providing a material having a desirable modulus.

Additional implant structures can also be crosslinked as described herein. For example, a component can include a polymeric rod within a collar. The polymeric rod can have its surface crosslinked to prevent against wear caused by relative motion between the polymeric rod and the collar.

The above-disclosed subject matter is to be considered illustrative, and not restrictive, and the appended claims are intended to cover all such modifications, enhancements, and other embodiments that fall within the true scope of the present invention. For example, it is noted that the components in the exemplary embodiments described herein are referred to as “superior” and “inferior” for illustrative purposes only and that one or more of the features described as part of or attached to a respective half can be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. A method of treating a patient, the method comprising:
determining a patient characteristic associated with the patient;
determining a property value based at least in part on the patient characteristic; and
determining a crosslinking parameter based at least in part on the property value.

2. The method of claim 1, further comprising:
effecting crosslinking of a bulk polymeric material of a device component based at least in part on the crosslinking parameter.

3.-6. (canceled)

7. The method of claim 2, wherein effecting crosslinking of the device component includes irradiating the device component.

8.-9. (canceled)

10. The method of claim 1, wherein determining the patient characteristic includes reviewing a patient medical file associated with the patient.

11.-17. (canceled)

18. A method of forming an implant device component, the method comprising:
determining a configuration of an implant device component; and
effecting crosslinking in a portion of a bulk polymeric material of the implant device component.

19. The method of claim 18, further comprising treating the implant device component.

20. The method of claim 19, wherein treating the implant device component includes sterilizing the implant device component.

21. The method of claim 19, wherein treating the implant device component includes annealing the bulk polymeric material of implant device component.

22. The method of claim 19, wherein treating the implant device component includes surface treating the implant device component.

23. The method of claim 18, wherein effecting crosslinking in the portion of the bulk polymeric material of the implant device component includes irradiating the portion of the bulk polymeric material.

24. The method of claim 23, wherein effecting crosslinking in the portion of the bulk polymeric material includes masking irradiation from a second portion of the bulk polymeric material.

25. The method of claim 18, wherein effecting crosslinking in the portion of the bulk polymeric material of the implant device component includes forming a temperature gradient in the bulk material.

26. (canceled)

27. The method of claim 18, wherein the implant device component includes a nucleus of a spinal implant device.

28. The method of claim 27, wherein the portion is an anterior portion of the nucleus and wherein effecting crosslinking in the portion includes crosslinking the anterior portion of the nucleus.

29. The method of claim 27, wherein the portion is a posterior portion of the nucleus and wherein effecting crosslinking in the portion includes crosslinking the posterior portion of the nucleus.

30. The method of claim 27, wherein the portion is a center portion of the nucleus and wherein effecting crosslinking in the portion includes crosslinking the center portion of the nucleus.

31.-47. (canceled)

48. A prosthetic device comprising:
a component configured to be interposed between two osteal structures, the component formed of a bulk polymeric material including a first portion of the bulk polymeric material crosslinked to a greater extent than a second portion of the bulk polymeric material.

49. The prosthetic device of claim 48, wherein the two osteal structures include an inferior vertebra and a superior vertebra.

50. The prosthetic device of claim 48, wherein the component is configured to be interposed within a region surrounded by an annulus fibrosis and between an inferior vertebra and a superior vertebra.

51.-53. (canceled)

54. The prosthetic device of claim 48, wherein the first portion is a center portion.

55. The prosthetic device of claim 48, wherein the first portion is an end portion.

56. The prosthetic device of claim 48, wherein the component has a maximum radius between about 3 mm and about 15 mm.

57. (canceled)

58. A kit comprising:
a prosthetic device comprising a bulk polymeric material; and
instructions relative to crosslinking the bulk polymeric material.
